(19) United States
(12) Patent Application Publication
(10) Pub. No.: US 2026/0022315 A1
(43) Pub. Date: Jan. 22, 2026

(54) NOVEL METHOD OF PRODUCING AN ALGAE COMPOSITION

(71) Applicant: ENLIGHTENED SOIL CORP, Johns Island, SC (US)

(72) Inventors: George Jesse TAYLOR, IV, Johns Island, SC (US); Christopher SPAULDING, Johns Island, SC (US); Andrew SHULER, Charleston, SC (US); Steve MORTON, Hanahan, SC (US)

(21) Appl. No.: 18/997,155

(22) PCT Filed: Jul. 20, 2023
(86) PCT No.: PCT/US2023/070624

§ 371 Date: Jan. 20, 2025

Related U.S. Application Data
(63) Continuation of application No. 17/869,140, filed on Jul. 20, 2022.

(51) Int. Cl. C12M 1/00; A01G 33/00; C12M 1/12; C12N 1/12

(52) U.S. Cl. CPC C12M 21/02; A01G 33/00; C12M 23/06; C12M 31/10; C12M 43/06; C12N 1/12

(57) ABSTRACT

A system and method for growing microalgae capable of mixotrophic metabolism, preferably Chlorella sp. Microalgae grown in the system using the method are able to survive and grow in dark refrigeration, which allows the algae to be stored and transported for application as a live culture. In addition, the microalgae can be grown in sufficient quantities to be sold commercially for application to crops as a biostimulant.

### BACKGROUND OF THE INVENTION

Rapid population growth following World War II prompted concerns about food security, a limit on the earth's ability to feed growing numbers. Malthusians predicted famine, possibly by the 1990s. This did not happen because of the Green Revolution, an unexpected increase in agricultural efficiency and yield. One element of this was the development of nitrogenous fertilizers, which was in turn a spin-off from munitions technology. Gunpowder factories became fertilizer factories (swords to plowshares).

Synthetic chemical fertilizers make plants grow. However, after seven decades of use there have been unintended consequences such as: 1) declining soil fertility and crop yield stagnation, 2) increasing greenhouse gases, and 3) contamination of groundwater leading to wild algae bloom. The production of synthetic fertilizers requires burning methane; it is estimated that 12% of greenhouse gases originate with modern agriculture, much of that from the use of chemical fertilizers.

In nature, plant growth depends on a symbiotic relationship between plants and soil microorganisms. Plants exude sugars (carbon compounds) from their roots that feed microorganisms present in the soil, in particular bacteria. In return, the soil microorganisms process nutrients needed for plant growth. Soil bacteria convert atmospheric nitrogen to ammonium compounds that can be absorbed by plant roots. This natural process is known as nitrogen fixation, and is primarily a function of bacteria in close contact with roots (this area is known as the “rhizosphere”), and not the work of the plants. Fertile soil in the rhizosphere has as many as 10 billion bacteria per gram. Microbial composition and activity in the soil defines “fertility”. Crop rotation is done with legumes to add nitrogen to soil, but it is the bacteria in legume root nodules that fix nitrogen. Other bacteria solubilize phosphorous that is soil-bound.

Chemical fertilizer bypasses the natural process. Over time, microbial activity—fertility—and levels of soil nitrogen and carbon decline. Chemical fertilizers add macro-nutrients, nitrogen, phosphorous and potassium, to the soil in forms that can be directly absorbed by roots. However, plants absorb just 30% of the nitrogen applied through chemical fertilization. Much of the rest goes into solution, eventually reaching groundwater. It is well-known that the addition of fertilizer to waterbodies, such as is known to occur through farm field runoff into ponds and lakes, promotes the growth of wild algae, leading to harmful algal blooms. In addition, some nitrogen in the fertilizer is converted to nitrous oxide, a potent greenhouse gas. Globally, agriculture accounts for 75% of nitrous oxide released in the atmosphere.

Composted organic material such as manure can be applied to soil to provide nitrogen as well as bacteria and carbon that feeds microbes. However, use of compost is inconvenient because of bulk, and is hard to apply at scale. Processing manure to make organic fertilizers is an industrial process, and there are energy costs with shipping.

Biostimulants provide another way to promote plant growth. Defined in the 2018 U.S. Farm bill, a plant biostimulant (PBS) is a compound or organism that promotes natural processes. A large body of research has shown that live microalgae, in particular Chlorella vulgaris, are effective biostimulants through both foliar and soil application. Furthermore, the stimulant effect of microalgae on soil microorganisms is regenerative: application of the microalgae increases organic matter, bacterial mass and soil respiration (a measure of bacterial activity). In other words, application of microalgae improves soil fertility. However, in the past, it was not possible to use live microalgae as biostimulants in large scale, because practicable storage of live microalgae was not available and live microalgae had to be produced on-site and used immediately after harvest.

### SUMMARY OF THE INVENTION

The present invention is a novel approach to inducing heterotrophic metabolic activity in microalgae, meaning that the microalgae grown using the method can consume organic material from medium to grow if placed under conditions where photosynthesis is not possible, i.e., where there is no light. Embodiments of the present invention are directed at improving a method used for growing Chlorella sp., preferably Chlorella vulgaris, in sufficient quantities to be sold commercially for application to crops as a biostimulant after the algae harvested. In particular, the microalgae grown using the improved method are able to survive dark refrigerated storage and shipment.

Microalgae, and in particular Chlorella vulgaris, has been grown for commercial use for decades. A known method for growing microalgae such as C. vulgaris at commercial volumes involves photobioreactors (PBR) to maximize growth in limited space.

Most commercial uses of microalgae do not involve live microalgae. For example, when microalgae are grown for oil content (biofuel), or used as an animal feed additive, the algae are processed immediately upon harvesting. Commercially available algae-based biostimulants include cell-free algae extracts that contain growth signaling compounds (phytohormones) such as auxins and cytokinins that stimulate plant growth and also promote soil fertility. Others are growth media from algae cultivation that contain the same growth signaling compounds secreted by algae during their growth. Importantly, none of the commercially available biostimulants contain live green algae cells. Most biostimulants are marketed as supplements to chemical fertilizer; they are not potent enough to replace it. Early university research showed that application of live algae-grown on site and applied at the time of algae harvest-provide enough boost to yield and fertility that chemical fertilizer could be eliminated. The live cell algae biostimulant proved more effective than algae derivatives. For this reason, a method to preserve live algae during shipment and storage could lead to reduced need for synthetic chemical fertilizer.

A known commercially available biostimulant comprising live cells uses cyanobacteria (“blue-green algae”, which actually is a bacterium). Cyanobacteria are an effective plant biostimulant, and produce the growth signaling compounds which are also produced by green algae. Like other bacteria, cyanobacteria can be dried, packaged, then reconstituted in water and applied as live cells. However, disadvantageously, over the last two decades neurotoxins (including β-N-methylamino-L-alanine, BMAA) produced by cyanobacteria have been related to an outbreak of a neurodegenerative illness. Because of this, there is a need for safer biostimulants based on live cells.

Microalgae grown by the method taught herein remain viable despite being bottled in the growth medium from the PBR as an “algae concentrate” and stored under refrigeration (6° C.). Refrigeration keeps the concentrate free of contaminants during storage and transport to the site of application because it inhibits growth of common contaminants including protozoans and bacteria. Refrigerated storage is typically dark, which creates a problem for microalgae, because, like higher plants, microalgae are usually autotrophic, meaning that they maintain viability and grow through generation of nutrients by light-dependent photosynthesis:

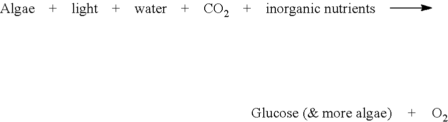

When microalgae are deprived of light photosynthesis stops and the algal cell count falls rapidly (in our laboratory, as much as 50% in five days, then with minimal recovery, Table 1). Thus, with standard methods of propagation, live microalgae have to be produced locally for immediate application as a PBS. This is problematic for widespread commercial use since microalgae are not easy to grow. Studies showing that live microalgae, i.e., locally grown and applied immediately following harvest, are an effective PBS have been done in northern Africa, southeast Asia, and eastern Europe, but the method has not been adopted for commercial use because it is difficult to replicate. Without wishing to be bound by theory, the assumed benefit of live algae, in particular Chlorella vulgaris, is the continued production of signaling compounds after their application to plants or soil. Another beneficial effect of live algae, in particular Chlorella vulgaris cells is that they were observed to ingest soil bacteria—probably through phagocytosis—transport them into plant root hairs, and finally release them into the interstitial space where they function as beneficial endophytes. There has not been a method for growing and storing live microalgae at scale so that it can be grown at one location and distributed to other distant locations for application without colony degradation. The essence of the present invention is a method for growing microalgae, and in particular Chlorella vulgaris, that remain viable without a significant decline in cell count while in cold, dark storage due to the induction of heterotrophic metabolism; i.e., microalgae grown by this method are able to consume organic material and oxygen and grow in the absence of light. This is achieved through hyperoxygenation of the water used in the growth medium in the PBR. In the hyperoxygenated environment the microalgae alter metabolic behavior and become “mixotrophic,” meaning that though they continue to produce food/energy with photosynthesis while there is light (autotrophic metabolism), they become capable of heterotrophic growth, able to consume organic nutrients, while in the dark. It has been known that algae are capable of heterotrophic feeding, as it has been observed to be induced by the addition of sugars like glucose to growth medium, in particular when exposed to high concentrations of glucose. The present method induces heterotrophic metabolism by hyperoxygenation of the growth media, i.e. by saturating the growth media with oxygen nanobubbles. This has not been reported previously. Nor have there been prior reports of microalgae capable of maintaining algae colony cell count and viability while in dark storage. While this method has been described for growing C. vulgaris, it would apply to all Chlorella sp. as well as other microalgae capable of mixotrophic metabolism.

Currently, PBRs are used to grow microalgae in water supplemented with inorganic nutrients. Microalgae grown in this manner can be used processed immediately upon harvest but cannot be transported for application for any use requiring live microalgae cells since the microalgae do not survive dark, refrigerated storage. Growing algae in photobioreactors has been common practice. In addition, the use of nanobubble generators as a part of these systems has been used to produce ozone nanobubbles for sterilization of algae growth media, and to clean algae growing equipment (tanks and plumbing). The improvement described herein comprises an oxygen concentrator having an attached nanobubble generator (NBG). In other words, the present invention provides, for the first time, a photobioreactor that is equipped with means for supplying oxygen in the form of nanobubbles. As illustrated in FIG. 1, the inventive system includes an NBG positioned to receive sterilized water from a source. The sterilized water is fed through the NBG to receive nanobubbles of oxygen to increase the measurable oxygen concentration of the water to a saturation of approximately 500%, i.e., a state of hyperoxygenation.

The hyperoxygenated water is then pumped to fill at least one PBR. A well-known mixture of inorganic nutrients widely used for algae production is added to the sterilized water in the PBR to create the growth medium. An example for a typical medium that can be used for growing the algae according to the present invention is commercially available Guillard's/F/₂ medium. Preferably, in the method of the present invention, there is no externally added carbohydrate present in the culture medium. Then, the PBR is inoculated with microalgae.

The PBRs used in the system are constructed from translucent material so that light from outside the PBR can be used by the microalgae cells growing inside the PBR for photosynthesis. An artificial light source is mounted outside the PBR and simulates a 24-hour day cycle. Since photosynthesis also requires carbon dioxide, a standard aquarium stone bubbler is used to introduce filtered ambient air into the growth medium to provide carbon dioxide and to keep the microalgae cells mixed and in suspension. Upon harvest from the PBR, the microalgae cells are bottled with their growth medium as algae concentrate containing a minimum of 10 million algae cells per mL; this concentrate is the final product that can be diluted and applied as a plant and soil biostimulant.

An important feature of the improved method is that the microalgae cells it produces are capable of surviving in dark, refrigerated storage for more than six months, even as long as eighteen months, which is enough time to allow for transport to an agricultural site for application. The bottled algae concentrate is placed directly into refrigeration (6° C.) for storage prior to sale for use as a biostimulant. However, refrigeration is not essential, since algae can grow over a wide range of temperatures. Refrigeration, however, advantageously prevents growth of bacterial and protozoan contaminants if storage is prolonged beyond two months. If the product is used within that time, refrigeration is unnecessary. Contamination leading to loss of the algae colony during the initial two months of storage has been rare, probably because heterotrophic feeding suppresses contaminant growth. The compositions of the present invention that contain live algae can be stored at refrigerator temperature, e.g. at 6° C., for more than eight months, although the recommended use-by date is six months from harvest. The microalgae grown in the system using this method not only remain viable but are able to continue growing while in storage, which maintains the cell count necessary for application for use as an effective biostimulant (see Table 1). The application rate of live algae is 50,000 cells per square foot, applied to soil, foliage or both; as such, one liter of algae concentrate can be diluted to treat 4.5 acres; 5 ml of concentrate treats 1,000 square feet.

Another feature of the improved method is that it allows microalgae to be grown at scale for agricultural use at low cost, requiring minimal engineering, and providing high yield of microalgae in a small space. In particular, the present invention allows growing the algae in liter photobioreactor tanks of up to 750 liter volume. Previously, there was a concern that the increased diameter of the tank would create shading of the algae in the center of the tank, and thus hamper growth due to inefficient exposure to light. However, the present inventors have surprisingly found that algae cell counts in the larger tank rises as quickly as counts in a 300-liter PBR with its smaller diameter. One explanation for this is constant mixing of the algae with the air bubbler. This prevents algae cells from collecting in the middle of the tanks, further from the light source. Another explanation is that the algae are mixotrophic, i.e. they are not dependent on photosynthesis, so they continue to grow even in areas of the reactor which are more shaded or even in the dark. Use of the larger PBR tanks introduces increased efficiency and productivity.

In the context of the present invention, at least the following embodiments E1 to E72 are described:
E1. According to a first embodiment, the invention relates to a method of producing an algae composition that comprises viable algae and a liquid, comprising the step(s) of
   adding oxygen to the liquid to provide for a hyperoxygenated liquid;
   culturing the algae in the hyperoxygenated liquid at least temporarily under light exposure to allow propagation of the algae.
E2. The method of embodiment 1 comprising the steps of
   providing a liquid;
   adding oxygen to the liquid to provide for a hyperoxygenated liquid;
   inocculating the hyperoxygenated liquid with algae;
   culturing the algae in the hyperoxygenated liquid at least temporarily under light exposure to allow propagation of the algae.
E3. The method of embodiment 1 comprising the steps of
   providing a liquid comprising algae;
   adding oxygen to the liquid to provide for a hyperoxygenated liquid and/or adding hyperoxygenated liquid;
   culturing the algae in the hyperoxygenated liquid at least temporarily under light exposure to allow propagation of the algae.
E4. The method of any one of embodiments 1 to 3, wherein the viable cell count in the liquid is between 1 and 20 million cells/mL, preferably between 2 and 18 million cells/mL, more preferably between 5 and 15 million cells/mL, even more preferably between 7 and 14 million cells/mL, still more preferably between 10 and 13 million cells/mL, most preferably between 11 and 13 million cells/mL.
E5. The method of any one of embodiments 1 to 4, wherein a decay in viable cell count is at most 90%, preferably at most 80%, more preferably at most 70%, even more preferably at most 60%, still more preferably at most 50%, still more preferably at most 40%, still more preferably at most 30%, still more preferably at most 20%, still more preferably at most 10%, still more preferably at most 5% of the decay in viable cell count of a control culture of the same algae which is not hyperoxygenated within the same period of time.
E6. The method of any one of embodiments 1 to 5, wherein there is at most 10%, preferably at most 8%, more preferably at most 6%, even more preferably at most 4%, still more preferably at most 2%, still more preferably at most 1%, most preferably no decay in viable cell count within a time period of
   (i) between 1 and 24 months, preferably between 1 and 20 months, more between 1 and 18 months, even more preferably between 2 and 15 months, still more preferably between 2 and 12 months, even more preferably between 2 and 10 months, even more preferably between 3 and 9 months, most preferably between 4 and 8 months; or
   (ii) between 1 and 30 days, preferably 1 and 25 days, more preferably 1 and 15 days, even more preferably 1 and 10 days, most preferably 4 and 6 days; or
   (iii) at least 1 month, preferably at least 2 months, more preferably at least 3 months, even more preferably at least 4 months, still more preferably at least 5 months, even more preferably at least 6 months, still more preferably at least 7 months, most preferably at least 8 months;
when compared to a control culture of the same algae which is not hyperoxygenated.
E7. The method of any one of embodiments 1 to 6, wherein the viably cell count increases by at least 1%, preferably at least 5%, more preferably at least 10%, even more preferably at least 20%, still more preferably at least 30%, most preferably at least 40% within a time period of
   (i) between 1 and 30 days, preferably 5 and 25 days, more preferably 10 and 25 days, even more preferably 15 and 25 days, most preferably 18 and 22 days, or
   (ii) of between 1 and 24 months, preferably between 1 and 20 months, more between 1 and 18 months, even more preferably between 2 and 15 months, still more preferably between 3 and 12 months, even more preferably between 3 and 10 months, even more preferably between 3 and 8 months, still more preferably between 3 and 6 months, most preferably between 3 and 4 months,
when compared to a control culture of the same algae which is not hyperoxygenated.
E8. The method of any one of embodiments 1 to 7, wherein the viable algae can be stored at a temperature of between 0° C. and 10° C., preferably between 2° C. and 8° C., more preferably between 5° C. and 7° C. for at least 1 month, preferably at least 2 months, more preferably at least 4 months, even more preferably at least 5 months, still more preferably at least 6 months, even more preferably at least 7 months, even more preferably at least 8 months, most preferably as long as 18 months and/or wherein the viable algae can be stored at room temperature, preferably at a temperature of between 10° C. and 30° C., preferably between 15° C. and 25° C., more preferably between 18° C. and 22° C. for at least 1 week, preferably at least 2 weeks, more preferably at least 3 weeks, even more preferably at least 4 weeks, stil more preferably at least 5 weeks, even more preferably at least 6 weeks, still more preferably at least 7 weeks, most preferably at least 8 weeks, in particular at least 2 months without significant decay in viable cell count compared to the viable cell count at the time of harvesting the algae.
E9. The method of any one of embodiments 1 to 8, wherein oxygen is added only once.
E10. The method of any one of embodiments 1 to 9, comprising more than one step of adding oxygen to the liquid to provide for a hyperoxygenated liquid and/or adding hyperoxygenated liquid.
E11. The method of any one of embodiments 1 to 8, wherein oxygen is added continuously to the liquid.
E12. The method of any one of embodiments 1 to 11, wherein the method is carried out in a photobioreactor.
E13. The method of any one of embodiments 1 to 12, wherein exposing the algae in the liquid to light is carried out for at least 1 to 24 hours at 1 to 12 hour intervals, preferably at least 16 hours at 8 hour intervals.
E14. The method of any one of embodiments 1 to 13, wherein exposing the algae in the liquid to light is carried out at a wavelength of 200 nm to 800 nm, preferably 250 nm to 650 nm, more preferably 300 to 550 nm, even more preferably 400 to 500 nm, most preferably 440 nm.
E15. The method of any one of embodiments 1 to 14, wherein exposing the algae in the liquid to light is carried out at a power of 1 to 20 W, preferably 5 to 20 W, more preferably 10 to 20 W, even more preferably 10 to 15 W, most preferably 13 W.
E16. The method of any one of embodiments 1 to 15, wherein exposing the algae in the liquid to light is carried out at a light intensity of between 1,000 and 20,000 lux, preferably 5,000 and 15,000 lux, more preferably of between 8,000 and 12,000 lux.
E17. The method of any one of embodiments 1 to 16, wherein the method further comprises
   (i) monitoring the viable cell count; and/or
   (ii) harvesting the algae; and/or
   (iii) concentrating the harvested algae; and/or
   (iv) storing the algae.
E18. The method of any one of embodiments 1 to 17, wherein the viable algae are stored (i) at a temperature of between 0° C. and 10° C., preferably between 2° C. and 8° C., more preferably between 5° C. and 7° C.; and/or
   (ii) at a temperature of between 10° C. and 30° C., preferably between 15° C. and 25° C., more preferably between 18° C. and 22° C.; and/or
   (iii) in the dark.
E19. The method of any one of embodiments 1 to 18, wherein oxygen is added to the liquid by supplying oxygen nanobubbles to the liquid.
E20. The method of any one of embodiments 1 to 19, wherein the liquid has an oxygen level of at least 20 ppm, preferably at least 25 ppm, more preferably at least 30 ppm, even more preferably at least 35 ppm, still more preferably at least 40, even more preferably at least 45 ppm, most preferably at least 50 ppm.
E21. The method of any one of embodiments 1 to 20, wherein the liquid has an oxygen saturation of at least 100%, preferably of at least 200%, more preferably at least 300%, even more preferably at least 400%, most preferably at least 500%.
E22. The method of any one of embodiments 2 to 21, wherein the hyperoxygenated liquid which is inocculated with the algae has an oxygen level of at least 50 ppm and/or an oxygen saturation of at least 500%.
E23. The method of any one of embodiments 1 to 22, wherein the liquid is an aqueous solution, preferably selected from water, fresh water, sea water, sterilized water, culture medium and/or buffer.
E24. The method of any one of embodiments 1 to 17, wherein the liquid is a culture medium, preferably the culture medium comprising
   a) water, preferably sterilized water;
   b) nitrate, preferably an alkalimetal salt thereof, more preferably sodium nitrate;
   c) dihydrogenphosphate preferably an alkalimetal salt thereof, more preferably sodium dihydrogenphosphate;
   d) silicate, preferably an alkalimetal salt thereof, more preferably sodium silicate;
   e) one or more trace metals, preferably inorganic salts thereof, more preferably the trace metals are selected from cobalt, copper, iron, manganese, molybdenum and/or zinc; and/or
   f) one or more vitamins, preferably selected from vitamin B₁₂, biotin, and/or thiamine.
E25. The method of embodiments 1 to 24, wherein there is no externally added carbohydrate present in the liquid.
E26. The method of any one of embodiments 1 to 25, wherein no additional carbohydrate is added to the liquid to induce heterotrophic metabolism in the algae.
E27. The method according to any one of embodiments 1 to 26, operated in batch mode, fed-batch mode, semi-continuous mode or continuous mode.
E28. The method of any one of embodiments 1 to 27, wherein the algae are capable of mixotrophic metabolism, preferably wherein the algae are capable of both autotrophic and heterotrophic metabolism, even more preferably the algae have autotrophic metabolism when grown under light exposure and are capable of heterotrophic metabolism in the absence of light.
E29. The method of any one of embodiments 1 to 28, wherein the algae are obligate mixotroph, obligate autotroph and facultative heterotroph, facultative autotroph and obligate heterotroph, and/or facultative mixotroph.
E30. The method of any one of embodiments 1 to 29, wherein the algae are selected from the group of unicellular algae, preferably green algae, more preferably the algae are Chlorella, even more preferably the algae are Chlorella vulgaris.
E31. A composition comprising viable algae and a liquid, wherein the composition is obtainable by a method according to any one of embodiments 1 to 30.
E32. A composition comprising viable algae and a liquid, wherein the liquid is hyperoxygenated.
E33. The composition of embodiment 31 or 32, wherein the liquid comprises oxygen nanobubbles, preferably wherein the liquid is saturated with oxygen nanobubbles.
E34. The composition of any one of embodiments 31 to 33, wherein the liquid has an oxygen level of at least 20 ppm, preferably at least 25 ppm, more preferably at least 30 ppm, even more preferably at least 35 ppm, still more preferably at least 40, even more preferably at least 45 ppm, most preferably at least 50 ppm.
E35. The composition of any one of embodiments 31 to 34, wherein the liquid has an oxygen saturation of at least 200%, preferably at least 300%, more preferably at least 400%, most preferably at least 500%.
E36. The composition of any one of embodiments 31 to 35, wherein the liquid is selected from an aqueous solution, preferably selected from water, fresh water, sea water, sterilized water, culture medium and/or buffer.
E37. The composition of any one of embodiments 31 to 36, wherein the liquid is a culture medium comprising
   (a) water, preferably sterilized water;
   (b) nitrate, preferably an alkalimetal salt thereof, more preferably sodium nitrate;
   (c) dihydrogenphosphate preferably an alkalimetal salt thereof, more preferably sodium dihydrogenphosphate;
   (d) silicate, preferably an alkalimetal salt thereof, more preferably sodium silicate;
   (e) one or more trace metals, preferably inorganic salts thereof, more preferably the trace metals are selected from cobalt, copper, iron, manganese, molybdenum and/or zinc; and/or
   (f) one or more vitamins, preferably selected from vitamin B₁₂, biotin, and/or thiamine.
E38. The composition of embodiment 31 to 37, wherein there is no externally added carbohydrate present in the liquid.
E39. The composition of any one of embodiments 31 to 38, wherein no additional carbohydrate is added to the liquid.
E40. The composition of any one of embodiments 31 to 3, wherein the algae are capable of mixotrophic metabolism, preferably wherein the algae are capable of both autotrophic and heterotrophic metabolism, even more preferably the algae have autotrophic metabolism when grown under light exposure and are capable of heterotrophic metabolism in the absence of light.
E41. The composition of any one of embodiments 31 to 40, wherein the algae are obligate mixotroph, obligate autotroph and facultative heterotroph, facultative autotroph and obligate heterotroph, and/or facultative mixotroph.
E42. The composition of any one of embodiments 31 to 41, wherein the algae are selected from the group of unicellular algae, preferably green algae, more preferably the algae are Chlorella, even more preferably the algae are Chlorella vulgaris.
E43. The composition of any one of embodiments 31 to 42, wherein the viable cell count in the liquid is between 1 and 20 million cells/mL, preferably between 2 and 18 million cells/mL, more preferably between 5 and 15 million cells/mL, even more preferably between 7 and 14 million cells/mL, still more preferably between 10 and 13 million cells/mL, most preferably between 11 and 13 million cells/mL.
E44. The composition of any one of embodiments 31 to 43, wherein a decay in viable cell count is at most 90%, preferably at most 80, more preferably at most 70%, even more preferably at most 60%, still more preferably at most 50%, still more preferably at most 40%, still more preferably at most 30%, still more preferably at most 20%, still more preferably at most 10%, still more preferably at most 5%, when compared to the decay in viable cell count of a control culture of the same algae which is not hyperoxygenated in the same period of time.
E45. The composition of any one of embodiments 31 to 44, wherein there is at most 10%, preferably at most 8%, more preferably at most 6%, even more preferably at most 4%, still more preferably at most 2%, still more preferably at most 1%, most preferably no decay in viable cell count within a time period of
   (i) between 1 and 24 months, preferably between 1 and 20 months, more between 1 and 18 months, even more preferably between 2 and 15 months, still more preferably between 2 and 12 months, even more preferably between 2 and 10 months, even more preferably between 3 and 6 months, most preferably between 3 and 5 months; or
   (ii) between 1 and 30 days, preferably 1 and 25 days, more preferably 1 and 15 days, even more preferably 1 and 10 days, most preferably 4 and 6 days; or
   (iii) at least 1 month, preferably at least 2 months, more preferably at least 3 months, even more preferably at least 4 months, still more preferably at least 5 months, even more preferably at least 6 months, still more preferably at least 7 months, most preferably at least 8 months;
when compared to a control culture of the same algae which is not hyperoxygenated.
E46. The composition of any one of embodiments 31 to 44, wherein the viably cell count increases.by at least 1%, preferably at least 5%, more preferably at least 10%, even more preferably at least 20%, still more preferably at least 30%, most preferably at least 40% within a time period of
   (i) between 1 and 30 days, preferably 5 and 25 days, more preferably 10 and 25 days, even more preferably 15 and 25 days, most preferably 18 and 22 days, or
   (ii) between 1 and 24 months, preferably between 1 and 20 months, more between 1 and 18 months, even more preferably between 2 and 15 months, still more preferably between 3 and 12 months, even more preferably between 3 and 10 months, even more preferably between 3 and 8 months, still more preferably between 3 and 6 months, most preferably between 3 and 4 months,
when compared to a control culture of the same algae which is not hyperoxygenated.
E47. Use of the composition of any one of embodiments 31 to 46 for improving plant growth.
E48. The use of embodiment 47, wherein the plants are selected from fruits, vegetables and/or crops, preferably the crops are agricultural plants grown for food or fiber.
E49. The use of embodiment 48, wherein the fruits are selected from fruit-bearing trees, berry bushes, and/or pineapples.
E50. The use of embodiment 48, wherein the vegetables are selected from garden vegetables, preferably tomatoes, potatoes, cucumbers, pepper, carrots, squash, and/or pumpkin.
E51. The use of embodiment 48, wherein the crops are selected from vegetable crops, sugar beets, corn, beans, hay, peanuts, cotton, hemp and/or tobacco.
E52. A method of maintaining or improving soil fertility and/or for improving plant growth comprising the step(s) of applying the composition of any one of embodiments 31 to 46 to soil and/or plants.
E53. The method of embodiment 52, wherein the soil is agricultural land, pastureland, a playing field, a golf course, and/or a civic green space.
E54. The method of embodiment 52 or 53, comprising the step of diluting the composition of any one of embodiments 25 to 37 before applying the composition to soil and/or plants.
E55. The method of any one of embodiments 52 to 54, wherein the composition is applied in an amount of between 10 and 100,000 cells per sq ft, preferably between 100 and 90,000 cells per sq ft, preferably between 1,000 and 80,000 cells per sq ft, more preferably between 10,000 and 70,000 cells per sq ft, still more preferably between 20,000 and 60,000 cells per sq ft, even more preferably between 30,000 and 55,000 cells per sq ft, still more preferably between 35,000 and 55,000 cells per sq ft, still more preferably between 40,000 and 55,000 cells per sq ft, even more preferably between 45,000 and 55,000 cells per sq ft, most preferably in an amount of 50,000 cells per sq ft.
E56. A photobioreactor comprising means for supplying oxygen in the form of nanobubbles.
E57. The photobioreactor of embodiment 56, wherein the means for supplying oxygen in the form of nanobubbles comprise means for providing oxygen, preferably an oxygen concentrator, and means for generating nanobubbles, preferably a nanobubble generator.
E58. The photobioreactor of embodiment 57, wherein the means for providing oxygen are operably connected to the means for generating nanobubbles.
E59. The photobioreactor of any one of embodiments 56 to 58, comprising one or more of
   a) one or more reaction vessels, preferably made from fiberglass;
   b) one or more reservoirs;
   c) one or more light sources, preferably LED light sources, more preferably a tubular LED grow light and/or LED bulbs;
   d) one or more means for supplying dissolved gas to the photobioreactor;
   e) an oxygen concentrator;
   f) piping; and/or
   g) one or more valves.
E60. The photobioreactor of any one of embodiments 56 to 59, comprising a reaction vessel, preferably wherein the reaction vessel is a container characterized by one or more of the following:
   (a) the container is liquid-impermeable;
   (b) the container is cylindrically-shaped;
   (c) the container has fixed side walls and bottom;
   (d) the container has a removable lid; and/or
   (e) the container is fabricated from a translucent material, preferably comprising fiberglass.
E61. The photobioreactor of any one of embodiments 56 to 60, comprising a light source, preferably a tubular LED grow light and/or a LED bulb, more preferably wherein the light source is characterized by one or more of the following:
   a) a wavelength of 200 nm to 800 nm, preferably 250 nm to 650 nm, more preferably 300 to 550 nm, even more preferably 400 to 500 nm, most preferably 440 nm; and/or
   b) a light intensity of between 1,000 and 20,000 lux, preferably 5,000 and 15,000 lux, more preferably of between 8,000 and 12,000 lux; and/or
   c) a power of 1 to 20 W, preferably 5 to 20 W, more preferably 10 to 20 W, even more preferably 10 to 15 W, most preferably 13 W; and/or
   d) the light source being positioned vertically and equidistant around the one or more reaction vessels, preferably the light source being positioned at a distance of 0.5 to 50 cm, preferably 1 to 10 cm, more preferably 2 to 8 cm, even more preferably 5 to 6 cm, most preferably 5 cm from the one or more reaction vessels; and/or
   e) the light source comprising a timer operable to switch on and off the light source, preferably the timer is set to cycle said light source on for at least 1 to 24 hours and off for at least 1 to 12 hours, preferably on for 16 hours and off for 8 hours.
E62. The photobioreactor of any one of embodiments 56-61, comprising one or more means for supplying dissolved gas to the one or more reaction vessels which is not the means for supplying oxygen in the form of nanobubbles, preferably, wherein the one or more means for supplying dissolved gas to the one or more reaction vessels comprises a pump operable to push dissolved gas into the one or more reaction vessels, tubing for the dissolved gas to pass through, and a check valve, more preferably, wherein the one or more means for supplying dissolved gas to the one or more reaction vessels is an aquarium stone bubbler.
E63. The photobioreactor of any one of embodiments 56 to 62, comprising piping, preferably wherein the piping is made from a polymer, preferably polyethylene, and/or stainless steel, preferably the piping is made from a combination of polyethylene and stainless steel.
E64. The photobioreactor of any one of embodiments 56 to 63 comprising piping, preferably wherein the piping is adapted to provide for fluid connection of the of the photobioreactor's components.
E65. A system comprising one or more photobioreactors according to any one of embodiments 56 to 64.
E66. The system of embodiment 65 comprising at least two photobioreactors, preferably wherein the photobioreactors are in fluid connection.
E67. The system of embodiment 66, wherein the photobioreactors are arranged in a photobioreactor array.
E68. The system of embodiment 66 or 67, wherein the photobioreactors are connected in parallel.
E69. The system of any one of embodiments 65 to 68, comprising valves, wherein the valves are operable to allow separation of said at least one photobioreactor from other photobioreactors by opening or closing said valves.
E70. The system of any one of embodiments 65 to 69, wherein the one or more photobioreactors are in fluid connection with a nanobubble generator.
E71. The system of any one of claims 65 to 70, comprising a reservoir in fluid connection with the one or more photobioreactors.
E72. The system of any one of claims 65 to 71, comprising a discharge line operably connected to the one or more photobioreactors to remove liquid from the one or more photobioreactors, preferably the discharge line is made from polyethylene.

These and other features and their advantages will be apparent to those skilled in the art of propagation of microalgae using photobioreactors from a careful reading of the Detailed Description of the Invention and of Illustrative Embodiments accompanied by the drawings.

### DETAILED DESCRIPTION OF THE INVENTION

According to a first aspect, the present invention relates to a method of producing an algae composition that comprises viable algae and a liquid, comprising the step(s) of
adding oxygen to the liquid to provide for a hyperoxygenated liquid;
culturing the algae in the hyperoxygenated liquid at least temporarily under light exposure to allow propagation of the algae.

The term “viable” algae refers to live algae or living algae. Culturing the algae in the hyperoxygenated liquid at least temporarily under light exposure to allow propagation of the algae refers to exposing the algae to light for a certain time, for example by irradiating a reaction vessel in a photobioreactor. Exposure to light, however, does not need to be continuously and there might be temporary phases without exposure to light. The algae have autotrophic metabolism when grown under light exposure and are capable of heterotrophic metabolism in the absence of light. In particular, exposure to light should not be continuous since algae grow more efficiently with circadian exposure, typically 16 hours with light and 8 hours without. The dark phase is when heterotrophic metabolism occurs. Propagation of the algae refers to cell growth and division of algae cells providing for an increase in cell count but may also comprise a steady state of dividing and/or living cells as well as dying cells. The algae can be cultured using typical conditions known to the skilled person for culturing algae, e.g. as described in Chapter 2.3 “Algal production” in the Manual on the Production and Use of Live Food for Aquaculture, Food and Agricultural Organization of the United Nations Fisheries Technical Paper 361, Rome 1996, ISBN 92-5-103934-8.

According to a specific embodiment, the method of the first aspect comprises the steps of
providing a liquid;
adding oxygen to the liquid to provide for a hyperoxygenated liquid;
inocculating the hyperoxygenated liquid with algae;
culturing the algae in the hyperoxygenated liquid at least temporarily under light exposure to allow propagation of the algae.

This way, a fresh algae culture can be provided.

According to another specific embodiment, the method of the first aspect comprises the steps of
providing a liquid comprising algae;
adding oxygen to the liquid to provide for a hyperoxygenated liquid and/or adding hyperoxygenated liquid;
culturing the algae in the hyperoxygenated liquid at least temporarily under light exposure to allow propagation of the algae.

In a specific embodiment of the method of the first aspect, one or more of the following applies:
a) the liquid has a viable cell count, wherein the viable cell count is between 1 and 20 million cells/mL, preferably between 2 and 18 million cells/mL, more preferably between 5 and 15 million cells/mL, even more preferably between 7 and 15 million cells/mL, still more preferably between 10 and 15 million cells/mL, most preferably between 12 and 14 million cells/mL;
b) a decay in viable cell count is at most 90%, preferably at most 80%, more preferably at most 70%, even more preferably at most 60%, still more preferably at most 50%, still more preferably at most 40%, still more preferably at most 30%, still more preferably at most 20%, still more preferably at most 10%, still more preferably at most 5% of the decay in viable cell count of a control culture of the same algae which is not hyperoxygenated in the same period of time;
c) there is at most 10%, preferably at most 8%, more preferably at most 6%, even more preferably at most 4%, still more preferably at most 2%, still more preferably at most 1%, most preferably essentially no decay in viable cell count within a time period of
   between 1 and 24 months, preferably between 1 and 20 months, more between 1 and 18 months, even more preferably between 2 and 15 months, still more preferably between 2 and 12 months, even more preferably between 2 and 10 months, even more preferably between 3 and 6 months, most preferably between 3 and 5 months
   or
   between 1 and 30 days, preferably 1 and 25 days, more preferably 1 and 15 days, even more preferably 1 and 10 days, most preferably 4 and 6 days
   or
   at least 1 month, preferably at least 2 months, more preferably at least 3 months, even more preferably at least 4 months, still more preferably at least 5 months, even more preferably at least 6 months, still more preferably at least 7 months, most preferably at least 8 months
   when compared to a control culture of the same algae which is not hyperoxygenated; and/or
d) the viably cell count increases.by at least 1%, preferably at least 5%, more preferably at least 10%, even more preferably at least 20%, still more preferably at least 30%, most preferably at least 40% within a time period of between 1 and 30 days, preferably 5 and 25 days, more preferably 10 and 25 days, even more preferably 15 and 25 days, most preferably 18 and 22 days,
   or
   of between 1 and 24 months, preferably between 1 and 20 months, more between 1 and 18 months, even more preferably between 2 and 15 months, still more preferably between 3 and 12 months, even more preferably between 3 and 10 months, even more preferably between 3 and 8 months, still more preferably between 3 and 6 months, most preferably between 3 and 4 months, when compared to a control culture of the same algae which is not hyperoxygenated.

It is contemplated that in context of the present invention where viable cell counts are compared between hyperoxygenated liquids that contain viable algae and non-hyperoxygenated liquids that contain viable algae, culturing conditions are the same, including but not limited to culture temperature, salinity, pH, culture ingredients, light/dark cycles etc. In preferred embodiments, the liquid has a viable cell count of between 12 and 14 million cells/mL, in particular at the time of harvesting the algae. In the same or further preferred embodiments, there is a decay in viable cell count of at most 5% of the decay in viable cell count of a control culture of the same algae which is not hyperoxygenated within the same period of time, preferably within 5 days after harvest. In the same or further preferred embodiments, there is at most 4% decay in viable cell count within a time period of 4 to 6 days. In the same or further preferred embodiments, there is essentially no decay in viable cell count within a period of time of at least 2 months, most preferably at least 8 months. Preferably, there is essentially no decay in viable cell count within a period of time of at least 2 months at room temperature, preferably at a temperature of between 10° C. and 30° C., preferably between 15° C. and 25° C., more preferably between 18° C. and 22° C. Further preferably, there is essentially no decay in viable cell count within a period of time of at least 8 months when refrigerated, in particular at a temperature of between 0° C. and 10° C., preferably between 2° C. and 8° C., more preferably between 5° C. and 7° C. Typically, at the time of harvest, there is observed a small drop in cell count, which is attributed to a shock due to the sudden change of environment, but the heterotrophic algae are able to begin recovery quickly and even propagate. For example, the viable cell count increases by at least 5% within a time period of between 18 and 22 days, in particular such as within 19 days. In another example, the viable cell count increases by at least 40% within a time period of between 3 and 4 months, such as in particular 119 days.

The skilled person is aware of methods to determine the viable cell count, such as, for example, using a hemocytometer or the plate count method (e.g. according to ASTM D5465) or an automated equivalent thereof such as the Electrical Sensing Zone Method of Enumerating and Sizing Single Cell Suspensions according to ASTM F2149-16. Electrical Sensing Zone instrumentation is commonly referred to as a Coulter counter. Other methods known to the skilled person include dye exclusion assays, colorimetric assays, fluorometric assays, luminometric assays, and flow cytometric assays.

In specific embodiments of the method according to the first aspect, oxygen is added only once initially. In the alternative, the method comprises one or more further steps of adding oxygen to the liquid to provide for a hyperoxygenated liquid and/or adding hyperoxygenated liquid. In a further alternative, oxygen is added continuously to the liquid.

In specific embodiments of the method according to the first aspect, the method is carried out in a photobioreactor.

In specific embodiments of the method according to the first aspect, exposing the algae in the liquid to light is carried out
a) for at least 1 to 24 hours at 1 to 12 hour intervals, preferably at least 16 hours at 8 hour intervals of light exposure;
b) at a wavelength of 200 nm to 800 nm, preferably 250 nm to 650 nm, more preferably 300 to 550 nm, even more preferably 400 to 500 nm, most preferably 440 nm;
c) at a power of 1 to 20 W, preferably 5 to 20 W, more preferably 10 to 20 W, even more preferably 10 to 15 W, most preferably 13 W and/or
d) a light intensity of between 1,000 and 20,000 lux, preferably 5,000 and 15,000 lux, more preferably of between 8,000 and 12,000 lux.

This way, algae are efficiently irradiated with light to support autotroph metabolism making use of photosynthesis. Also, algae have/need a circadian light cycle to be mixotroph, in particular to switch from autotrophic mode to heterotrophic mode. The light exposure protocol according to the present invention thus facilitates switching between autotrophic and heterotrophic modes.

In specific embodiments of the method according to the first aspect, the method further comprises
a) monitoring the viable cell count; and/or
b) harvesting the algae; and/or
c) concentrating the harvested algae; and/or
d) storing the algae.

In specific embodiments of the method according to any of the first to the fifth aspect, the method is operated in batch mode, fed-batch mode, semi-continuous mode or in continuous mode. This provides for flexible handling of the grown algae, simple adaptation to scale-up and different reactor types. A batch operation mode allows harvesting of the entire algae concentrate from a photobioreactor. However, the inventors found that it is more efficient to do partial harvests, i.e. to operate in semi-continuous mode, and take from 40%,to as much as 70% of a tank, and add hyper-oxygenated medium to refill the tank. The algae that were not harvested are then re-exposed to hyperoxygenated liquid (in particular culture medium and/or buffer) as they continue to grow. The cells grow fast enough to allow harvest at 4-5 day intervals. The partial harvest process is efficient because cleaning between partial harvests is not necessary for longer operation time. After 4-5 months the rate of rise in cell counts from a tank declines; at that time the ‘tired’ tank is taken offline, drained, cleaned and re-started.

According to a second aspect, the present invention relates to a composition comprising viable algae and a liquid, wherein the composition is obtainable by a method according to any one of the first to fifth aspect.

According to a third aspect, the present invention relates to a composition comprising viable algae and a liquid, wherein the liquid is hyperoxygenated.

In specific embodiments in any of the method according to the first aspect or in any of the compositions according to the second and third aspect, the viable algae can be stored
a) at a temperature of between 0° C. and 10° C., preferably between 2° C. and 8° C., more preferably between 5° C. and 7° C.; and/or
b) at a temperature of between 10° C. and 30° C., preferably between 15° C. and 25° C., more preferably between 18° C. and 22° C.; and/or
c) in the dark.

The viable algae can be stored at a temperature of between 0° C. and 10° C. and/or in the dark. The expression “storing in the dark” refers to keeping the viable algae in the dark, i.e. in the absence of light and/or under absolute or essentially complete exclusion of light. However, refrigeration is not essential, since algae can grow over a wide range of temperatures. Refrigeration, however, advantageously prevents growth of bacterial and protozoan contaminants. On the other hand, algae can be stored at room temperature as long as two months, which advantageously simplifies storage conditions and shipping. For more prolonged storage, i.e. storage for a period of time longer than two months from harvest, refrigeration is recommended, i.e. it is recommended to keep the algae at a temperature of between 0° C. and 10° C., preferably between 2° C. and 8° C., more preferably between 5° C. and 7° C., most preferably at 6° C.

In specific embodiments in any of the method according to the first aspect or in any of the compositions according to the second and third aspect, the viable algae can be stored at a temperature of between 0° C. and 10° C., preferably between 2° C. and 8° C., more preferably between 5° C. and 7° C. for at least 1 month, preferably at least 2 months, more preferably at least 4 months, even more preferably at least 5 months, still more preferably at least 6 months, even more preferably at least 7 month, still more preferably at least 8 months, most preferably as long as 18 months without significant decay in viable cell count compared to the viable cell count at the time of harvesting the algae. In other or the same specific embodiments, in any of the method according to the first aspect or in any of the compositions according to the second and third aspect, the viable algae can be stored at room temperature, preferably at a temperature of between 10° C. and 30° C., preferably between 15° C. and 25° C., more preferably between 18° C. and 22° C. for at least 1 week, preferably at least 2 weeks, more preferably at least 3 weeks, even more preferably at least 4 weeks, stil more preferably at least 5 weeks, even more preferably at least 6 weeks, still more preferably at least 7 weeks, most preferably at least 8 weeks, in particular at least 2 months without significant decay in viable cell count compared to the viable cell count at the time of harvesting the algae. Thus, the present invention allows simplified and easy filling, storing, shipping and use of the live algae and thus facilitates their large-scale use as biostimulants.

In specific embodiments in any of the method according to the first aspect or in any of the compositions according to the second and third aspect, the composition comprising viable algae and liquid, or the liquid comprising viable algae, respectively, is characterized by a viable cell count, wherein
a) the viable cell count is between 1 and 20 million cells/mL, preferably between 2 and 18 million cells/mL, more preferably between 5 and 15 million cells/mL, even more preferably between 7 and 14 million cells/mL, still more preferably between 10 and 13 million cells/mL, most preferably between 11 and 13 million cells/mL;
b) a decay in viable cell count is at most 90%, preferably at most 80%, more preferably at most 70%, even more preferably at most 60%, still more preferably at most 50%, still more preferably at most 40%, still more preferably at most 30%, still more preferably at most 20%, still more preferably at most 10%, still more preferably at most 5% of the decay in viable cell count of a control culture of the same algae which is not hyperoxygenated;
c) there is at most 10%, preferably at most 8%, more preferably at most 6%, even more preferably at most 4%, still more preferably at most 2%, still more preferably at most 1%, most preferably no decay in viable cell count within a time period of between 1 and 24 months, preferably between 1 and 20 months, more between 1 and 18 months, even more preferably between 2 and 15 months, still more preferably between 2 and 12 months, even more preferably between 2 and 10 months, even more preferably between 3 and 9 months, most preferably between 4 and 8 months
   or
   between 1 and 30 days, preferably 1 and 25 days, more preferably 1 and 15 days, even more preferably 1 and 10 days, most preferably 4 and 6 days
   or
   at least 1 month, preferably at least 2 months, more preferably at least 3 months, even more preferably at least 4 months, still more preferably at least 5 months, even more preferably at least 6 months, still more preferably at least 7 months, most preferably at least 8 months
   when compared to a control culture of the same algae which is not hyperoxygenated; and/or
d) the viably cell count increases.by at least 1%, preferably at least 5%, more preferably at least 10%, even more preferably at least 20%, still more preferably at least 30%, most preferably at least 40% within a time period of between 1 and 30 days, preferably 5 and 25 days, more preferably 10 and 25 days, even more preferably 15 and 25 days, most preferably 18 and 22 days,
   or
   of between 1 and 24 months, preferably between 1 and 20 months, more between 1 and 18 months, even more preferably between 2 and 15 months, still more preferably between 3 and 12 months, even more preferably between 3 and 10 months, even more preferably between 3 and 8 months, most preferably between 3 and 6 months, still more preferably between 3 and 6 months, most preferably between 3 and 4 months,
   when compared to a control culture of the same algae which is not hyperoxygenated.

It is contemplated that in context of the present invention where viable cell counts are compared between hyperoxygenated liquids that contain viable algae and non-hyperoxygenated liquids that contain viable algae, culturing conditions are the same, including but not limited to culture temperature, salinity, pH, culture ingredients, light/dark cycles etc. In preferred embodiments, the liquid has a viable cell count of between 12 and 14 million cells/mL, in particular at the time of harvesting the algae. In the same or further preferred embodiments, there is a decay in viable cell count of at most 5% of the decay in viable cell count of a control culture of the same algae which is not hyperoxygenated within the same period of time, in particular within 5 days after harvest. In the same or further preferred embodiments, there is at most 4% decay in viable cell count within a time period of 4 to 6 days. In the same or further preferred embodiments, there is essentially no decay in viable cell count within a period of time of at least 2 months, most preferably at least 8 months. Preferably, there is essentially no decay in viable cell count within a period of time of at least 2 months at room temperature, preferably at a temperature of between 10° C. and 30° C., preferably between 15° C. and 25° C., more preferably between 18° C. and 22° C. Further preferably, there is essentially no decay in viable cell count within a period of time of at least 8 months when refrigerated, in particular at a temperature of between 0° C. and 10° C., preferably between 2° C. and 8° C., more preferably between 5° C. and 7° C. Typically, at the time of harvest, there is observed a small drop in cell count, which is attributed to a shock due to the sudden change of environment, but the heterotrophic algae are able to begin recovery quickly and even propagate. For example, the viable cell count increases by at least 5% within a time period of between 18 and 22 days, in particular such as within 19 days. In another example, the viable cell count increases by at least 40% within a time period of between 3 and 4 months, such as in particular 119 days.

The skilled person is aware of methods to determine the viable cell count, such as, for example, using a hemocytometer or the plate count method (e.g. according to ASTM D5465) or an automated equivalent thereof such as the Electrical Sensing Zone Method of Enumerating and Sizing Single Cell Suspensions according to ASTM F2149-16. Electrical Sensing Zone instrumentation is commonly referred to as a Coulter counter. Other methods known to the skilled person include dye exclusion assays, colorimetric assays, fluorometric assays, luminometric assays, and flow cytometric assays. Also, cell viability is verfied by inspection such a microscopic inspection: live algae cells have a distinctive appearance with green protoplast, whereas non-viable cells appear as cell fragments.

In specific embodiments in any of the method according to the first aspect or in any of the compositions according to the second and third aspect, the liquid is hyperoxygenated. The term “hyperoxygenated” means oxygenated more than usually.

Molecular oxygen is poorly water soluble. In its aqueous solutions oxygen resides in (H2O)₂₀ clathrates. The solubility of oxygen in water is temperature- and pressure-dependent. At 25° C. and 101.3 kPa, freshwater contains about 6.04 mL/L of oxygen. Mineral salts in water decrease solubility of oxygen and, for instance, at 25° C. and 101.3 kPa seawater contains about 4.95 mL/L molecular oxygen. In context of the present invention, a hyperoxygenated liquid refers to a liquid that has a particularly high content of dissolved oxygen, preferably at the chemically and physically possible maximum. In other words, the hyperoxygenated liquid is hyper-saturated with dissolved oxygen. It is contemplated that the skilled person understands that the hyperoxygenated state of water varies with the ambient conditions. In context of the present invenvention, if not stated otherwise, the hyperoxygenated state refers to standard conditions, i.e. a temperature of 20° C. and a pressure of 1013 hPa. Without wishing to be bound by theory, the present inventors believe that hyperoxygenation induces heterotrophic metabolism in the algae which are capable of mixotrophism. Advantagously, hyperoxxygenation allows to induce heterotrophic metabolism in the algae even in the absence of any externally added carbohydrates. It is preferred that there is no externally added carbohydrate present in the liquid. Preferably, the amount of carbohydrates in the liquid is less than 10 wt.-%, preferably less than 8 wt.-%, more preferably less than 6 wt.-%, even more preferably less than 4 wt.-%, still more preferably less than 2 wt.-%, most preferably less than 1 wt.-% based on the total weight of the total culture medium. For example, when hyperoxygenation is used to induce heterotrophic metabolism in the algae, no additional carbohydrate needs to be added to induce heterotrophic metabolism in the algae. The liquid may continuously stay in hyperoxygenated state or the oxygen content of the liquid may decay over time. For example, freshly hyperoxygenated liquid in context of the present invention has an oxygen content of at least 50 ppm. After 48 hours in the photobioreactor, the liquid has an oxygen content of at least 30 ppm, and at the time of harvest at about 4 days, the liquid has an oxygen content of about 20 ppm.

In specific embodiments in any of the method according to the first aspect or in any of the compositions according to the second and third aspect, for adding oxygen to the liquid, oxygen nanobubbles are supplied to the liquid, and oxygen may be present as oxygen nanobubbles in the liquid. The term “nanobubbles” refers to gas bubbles having a diameter in the nanometer range, e.g. in the range of between 1 and 1,000 nm. Nanobubbles are usually characterized by a size distribution. Nanobubbles can be characterized by high-speed camera image analysis, electrical signal (Coulter counter), resonance mass, particle trajectory (Nanoparticle Tracking Analysis (NTA)), laser difraction, and dynamic light scattering (DLS) known to the skilled person, in particular DLS according to ASTM E3247-20. The dimensions of the bubbles in the nanometer range provides for unique physical, chemical and biological properties such as high total bubble surface area, strong negative surface charge providing for stability in solution minimizing off-gassing and/or coalescence. The oxygen nanobubbles of the invention can persist in solution for as long as 2 weeks under standard conditions. So, the algae concentrate harvested after 4-5 days of growth still has an elevated oxygen content. Nanobubbles facilitate increasing the concentration of dissolved gas in a liquid. Nanobubbles can be formed using any gas and injected into any liquid. In the context of the present invention, nanobubbles are preferably formed from oxygen that is injected into an aqueous liquid such as sterilized water, a culture medium or a buffer. Using oxygen nanobubbles allows to provide for high levels of dissolved oxygen (DO), which cannot be achieved by other, conventional aeration methods which do not make use of oxygen nanobubbles. Thus, oxygen nanobubbles provide for more efficient use of oxygen. Nanobubbles can be generated using a nanobubble generator.

In specific embodiments in any of the method according to the first aspect or in any of the compositions according to the second and third aspect, the liquid has an oxygen level of at least 20 ppm, preferably at least 25 ppm, more preferably at least 30 ppm, even more preferably at least 35 ppm, still more preferably at least 40, even more preferably at least 45 ppm, most preferably at least 50 ppm and/or the liquid has an oxygen saturation of at least 200%, preferably at least 300%, more preferably at least 400%, most preferably at least 500%. In preferred embodiments of the method according to the first aspect of the invention, in the step of inocculating the hyperoxygenated liquid with algae, the liquid is hyperoxygenated and has an oxygen level of at least 50 ppm and/or an oxygen saturation of at least 500%, preferably the liquid is hyperoxygenated using oxygen nanobubbles. The oxygen level or the oxygen saturation of a liquid, respectively, can be measured using any standard analysis method known to the person skilled in the art, for example, titration techniques, electrochemical analysis, e.g. using a Clark electrode or photochemical analysis. It is contemplated that corresponding sensors for determining oxygen levels in a liquid may be used. Without wishing to be bound by theory, the present inventors believe that hyperoxygenation or the oxygen level, e.g. of at least 50 ppm and/or an oxygen saturation of e.g. approximately 500% in the liquid, induces heterotrophic metabolism in the algae. This way, induction of heterotrophic metabolism in the algae, can advantageously be achieved even in the absence of any externally added carbohydrates in the liquid, preferably a culture medium and/or a buffer. For example, when hyperoxygenation is used to induce heterotrophic metabolism in the algae, no additional carbohydrate source needs to be added to the liquid.

In specific embodiments in any of the method according to the first aspect or in any of the compositions according to the second and third aspect, the liquid is an aqueous solution, preferably selected from water, fresh water, sea water, sterilized water, culture medium and/or buffer This way, the algae are provided with optimum supply for growth and/or maintaining viability.

In specific embodiments in any of the method according to the first aspect or in any of the compositions according to the second and third aspect, the liquid is a culture medium, preferably the culture medium comprising
a) water, preferably sterilized water;
b) nitrate, preferably an alkalimetal salt thereof, more preferably sodium nitrate;
c) dihydrogenphosphate preferably an alkalimetal salt thereof, more preferably sodium dihydrogenphosphate;
d) silicate, preferably an alkalimetal salt thereof, more preferably sodium silicate;
e) one or more trace metals, preferably inorganic salts thereof, more preferably the trace metals are selected from cobalt, copper, iron, manganese, molybdenmum and/or zink; and/or
f) one or more vitamins, preferably selected from vitamin B₁₂, biotin, and/or thiamine.

In some specific embodiments, no additional or external carbohydrate is added to the liquid to induce heterotrophic metabolism in the algae. As used herein, the term “carbohydrates” includes monosaccharides, such as glucose, fructose and/or galactose, disaccharides such as sucrose, maltose, trehalose and/or lactose, oligosaccharides such as raffinose and/or maltodextrines. By the expression “no additional or external carbohydrate is added” or “externally added carbohydrate” in context of the present invention, it is meant that no carbohydrate is added to the liquid which has not formed part of that liquid yet, in particular the culture medium, while, at the same time, it is not excluded that carbohydrates such as glucose may be formed during the culturing, e.g. by autotrophic algae that are either alive or dead and released into the liquid. An example for a typical medium that can be used for growing the algae according to the present invention is commercially available Guillard's/F/₂ medium.

In specific embodiments in any of the method according to the first aspect or in any of the compositions according to the second and third aspect, the algae are capable of mixotrophic metabolism, preferably wherein the algae are capable of both autotrophic and heterotrophic metabolism. In particular embodiments, the algae have autotrophic metabolism when grown under light exposure and are capable of heterotrophic metabolism in the absence of light. In particular embodiments, the algae are obligate mixotroph, obligate autotroph and facultative heterotroph, facultative autotroph and obligate heterotroph, and/or facultative mixotroph. This way, the algae are able to switch from autotrophism to heterotrophism and vice versa. Mixotroph algae can adapt their metabolism to ambient conditions. In context of the present invention, for example, the algae can switch from autotrophism during a growth or a culturing phase in a photobioreactor to heterotrophism when stored in the dark. Algae that are particularly useful in context of the present invention are selected from the group of unicellular algae, preferably green algae, more preferably the algae are Chlorella, even more preferably the algae are Chlorella vulgaris.

In an illustrative embodiment, a method according to the first aspect comprises:
Creating growth medium by adding nanobubbles of oxygen to sterilized water by feeding said water through a nanobubble generator to reach hyperoxygenation, or an oxygen level of at least 50 ppm, with an oxygen saturation of approximately 500%;
pumping said sterilized, hyperoxygenated water through tubing connected at one end to said nanobubble generator and at a second end to stainless steel piping plumbed into at least one photobioreactor to fill said photobioreactor with growth medium;
adding inorganic nutrient solution formulated to support microalgal growth to the at least one photobioreactor containing sterilized, hyperoxygenated water to create growth medium and inoculating said growth medium with a substantially homogenous monoculture of microalgae capable of mixotrophic metabolism, preferably Chlorella sp., having a concentration of 6-8 million cells/mL;
exposing the inoculated growth medium to light for 16 hours at 8 hour intervals;
continuously delivering ambient air through an aquatic stone bubbler carrying a filter placed inside the PBR near the base;
monitoring the growth rate of the microalgae by draining a certain volume of said growth medium from the PBR and counting the cells; and
harvesting the microalgae by draining said growth medium containing said microalgae grown to a concentration of 12 million cells/mL into containers, preferably composed of polyethylene, and storing said containers at 6° C.

According to an fourth aspect, the present invention relates to the use of a composition according to the second or third aspect for improving plant growth.

In preferred embodiments of the use according to the fourth aspect, the composition stimulates plants, preferably the plants are selected from fruits, vegetables and/or crops, preferably the crops are agricultural plants grown for food or fiber. Examples for fruits can be without limitations fruit-bearing trees, berry bushes, pineapples. Examples for vegetables can be without limitations garden vegetables, preferably tomatoes, potatoes, cucumbers, pepper, carrots, squash, and/or pumpkin. Examples for crops can be without limitations sugar beets, sugar cane corn, beans, hay, peanuts, tobacco cotton, hemp, and/or flax.

According to a fifth aspect, the present invention relates to a method of maintaining or improving soil fertility and/or for improving plant growth comprising the step(s) of
applying the composition according to the second or third aspect to soil and/or plants.

In specific embodiments of the method according to the ninth aspect, the soil can be agricultural land, pasture land, a playing field, a golf course, and/or a civic green space. Thus, the composition of the present invention can be applied to a broad spectrum of land. In further specific embodiments of the method according to the ninth aspect, the method comprises the step of diluting the composition of any one of claims 25 to 37 before applying the composition to soil and/or plants. In particular, the the composition is applied in an amount of between 10 and 100,000 cells per sq ft (108 and 1,080,000 cells per m²), preferably between 100 and 90,000 cells per sq ft (1,080 and 970,000 cells per m²), preferably between 1,000 and 80,000 cells per sq ft (10,800 and 860,000 cells per m²), more preferably between 10,000 and 70,000 cells per sq ft (108,000 and 750,000 cells per m²), still more preferably between 20,000 and 60,000 cells per sq ft (215,000 and 645,000 cells per m²), even more preferably between 30,000 and 55,000 cells per sq ft (323,000 and 592,000 cells per m²), still more preferably between 35,000 and 55,000 cells per sq ft (378,000 and 592,000 cells per m²), still more preferably between 40,000 and 55,000 cells per sq ft (431,000 and 592,000 cells per m²), even more preferably between 45,000 and 55,000 cells per sq ft (484,000 and 592,000 cells per m²), most preferably in an amount of 50,000 cells per sq ft (538,000 cells per m²). The composition and method of the present invention thus allows for efficient maintenance or improvement of soil fertility and/or plant growth.

According to a sixth aspect, the present invention relates to a photobioreactor comprising means for supplying oxygen in the form of nanobubbles.

In specific embodiments of the photobioreactor according to the sixth aspect, the means for supplying oxygen in the form of nanobubbles comprise means for providing oxygen, preferably an oxygen concentrator, and means for generating nanobubbles, preferably a nanobubble generator. Nanobubble generators allow for efficient and uniform production of nanobubbles, i.e. of nanobubbles of a particular size distribution.

In specific embodiments of the photobioreactor according to the sixth aspect, the photobioreactor comprises one or more of
a) one or more reaction vessels, preferably made from fiberglass;
b) one or more reservoirs;
c) one or more light sources, preferably LED light sources, more preferably a tubular LED grow light and/or LED bulbs;
d) one or more means for supplying dissolved gas to the photobioreactor;
e) an oxygen concentrator;
f) piping; and/or
g) one or more valves.

In specific embodiments, the photobioreactor comprises a reaction vessel, preferably wherein the reaction vessel is a container characterized by one or more of the following:
a) the container is liquid-impermeable;
b) the container is cylindrically-shaped;
c) the container has fixed side walls and bottom;
d) the container has a removable lid; and/or
e) the container is fabricated from a translucent material, preferably comprising fiberglass.

This way, a suitable reaction vessel for photobiosynthesis is provided. The reaction vessels of a photobioreactor according to the present invention can have any volume. A preferred volume is up to 750 liter. There was concern that the increased diameter of the tank would create shading of the algae in the center of the tank, and slow growth. However, the inventors of the present invention have surprisingly found that algae cell counts in the larger tank rises as quickly as counts in the 350 liter photobioreactor with its smaller diameter (under comparable conditions). One explanation for this is constant mixing of the algae with the air bubbler. This prevents algae cells from collecting in the middle of the tanks, further from the light source. Another explanation is that the algae are mixotrophic, so they continue to grow in the dark. They are not just dependent on photosynthesis. Use of the larger reaction vessel introduces increased efficiency and productivity.

In specific embodiments, the photobioreactor comprises preferably a tubular LED grow light, more preferably wherein the light source is characterized by one or more of the following:
a) a wavelength of 200 nm to 800 nm, preferably 250 nm to 650 nm, more preferably 300 to 550 nm, even more preferably 400 to 500 nm, most preferably 440 nm; and/or
b) a light intensity of between 1,000 and 20,000 lux, preferably 5,000 and 15,000 lux, more preferably of between 8,000 and 12,000 lux; and/or
c) a power of 1 to 20 W, preferably 5 to 20 W, more preferably 10 to 20 W, even more preferably 10 to 15 W, most preferably 13 W; and/or
d) the light source being positioned vertically and equidistant around the one or more reaction vessels, preferably the light source being positioned at a distance of 0.5 to 50 cm, preferably 1 to 10 cm, more preferably 2 to 8 cm, even more preferably 5 to 6 cm, most preferably 5 cm from the one or more reaction vessels; and/or
e) the light source comprising a timer operable to switch on and off the light source, preferably the timer is set to cycle said light source on for at least 1 to 24 hours and off for at least 1 to 12 hour, preferably on for 16 hours and off for 8 hours.

This way, the algae can efficiently be exposed to light during growth in the photobioreactor.

In specific embodiments, the photobioreactor comprises one or more means for supplying dissolved gas to the one or more reaction vessels, which is not the means for supplying oxygen in the form of nanobubbles, preferably, wherein the one or more means for supplying dissolved gas to the one or more reaction vessels comprises a pump operable to push dissolved gas into the one or more reaction vessels, tubing for the dissolved gas to pass through, and a check valve, more preferably, wherein the one or more means for supplying dissolved gas to the one or more reaction vessels is an aquarium stone bubbler. This way, for example ambient air or any other gas mixture can efficiently be supplied to a liquid in the photobioreactor, in particular to the reaction vessel of the photobioreactor.

In preferred embodiments, the photobioreactor comprises piping, preferably wherein the piping is made from a polymer, preferably polyethylene, and/or stainless steel, preferably the piping is made from a combination of polyethylene and stainless steel. This way, liquid can be passed through the photobioreactor. Stainless steel allows for safety, long equipment life since erosion is minimized and for easy sterilizing the equipment during maintenance. Preferably, the piping is adapted to provide for fluid connection of the photobioreactor's components, for example, fluid connection of the means for supplying oxygen in the form of nanobubbles, preferably the nanobubble generator, the one or more reaction vessels, the one or more reservoirs, one or more means for supplying dissolved gas to the photobioreactor, oxygen concentrator and/or the one or more valves. For example, a piping may provide for fluid connection of the reservoir with the reaction vessel and the means for supplying oxygen in the form of nanobubbles, and said piping may carry one or more valves, and eventually allow for filling the photobioreactor with sterilized, hyperoxygenated water or draining growth medium containing algae from said photobioreactor.

According to a seventh aspect, the present invention relates to a system comprising one or more photobioreactors according to the sixth aspect.

In specific embodiments, the system comprises at least two photobioreactors, preferably wherein the photobioreactors are in fluid connection and/or wherein the photobioreactors are arranged in a photobioreactor array. Also, the bioreactors are preferably connected in parallel. This way, the growth of algae can be scale-up easily since large amounts of algae can be cultured in parallel.

In specific embodiments, the system comprises valves, wherein the valves are operable to allow separation of said at least one photobioreactor from other photobioreactors by opening or closing said valves. This allows individual filling and/or drainage of a bioreactor in the system. The valves also allow for controlling passage to and from individual photobioreactors as well as between individual photobioreactors.

In specific embodiments, the one or more photobioreactors are in fluid connection with a nanobubble generator. This way, oxygen nanobubbles can be efficiently supplied to the one or more bioreactors. There can be one nanobubble generator for each of the one or more photobioreactors. In the alternative, there can be one nanobubble generator for the plurality of photobioreactors together. This way, supply with oxygen nanobubbles is centralized and the system is simplified. This allows for lower costs and easy maintenance of the system.

In preferred embodiments, the system comprises a reservoir in fluid connection with the one or more photobioreactors. The reservoir can contain a liquid, such as an aqueous solution, preferably selected from water, fresh water, sea water, sterilized water, culture medium and/or buffer, which is supplied to the one or more photobioreactors. One reservoir can feed individual photobioreactors or all of the photobioreactors of the system. A reservoir may, for example, be a holding tank. Preferably, the reservoir is positioned such way to allow gravity-feeding of the photobioreactors.

In specific embodiments, the system comprises a discharge line operably connected to the one or more photobioreactors to remove liquid from the one or more photobioreactors, preferably the discharge line is made from polyethylene. The discharge line facilitates harvesting of the algae and allows for continuous algae production.

In an illustrative embodiment, in a system according to the seventh aspect, the system comprising at least one reservoir, preferably a holding tank, a photobioreactor, a nanobubble generator, a light source, one or more means for supplying dissolved gas to the one or more reaction vessels,preferably an aquarium stone bubbler,
wherein said holding tank is connected through a valve carried on the tank to a first end of piping, said piping preferably comprising stainless steel, the second end of said piping connected to said nanobubble generator, preferably, said piping is connected to the photobioreactor near the base of said photobioreactor, said piping carrying a first valve positioned above said connection to said photobioreactor and a second valve positioned below said connection to said photobioreactor, for filling said photobioreactor with sterilized, hyperoxygenated water or draining growth medium containing microalgae from said photobioreactor through a length of said piping connected at a second end to a discharge line, preferably comprised of polyethylene tubing;
wherein said system comprises more than one photobioreactor arranged in a photobioreactor array, said photobioreactors are plumbed in parallel, such that said piping connected at said first end to said nanobubble generator and at said second end to a discharge line, carries additional lengths of piping, said additional piping connected to said additional photobioreactors near the base of each said additional photobioreactor, said additional piping carrying a first valve positioned above said connection to said additional photobioreactor and a second valve positioned below said connection to said additional photobioreactor, which valves allow separation of said at least one photobioreactor from other photobioreactors by opening or closing said valves.

It is contemplated that any embodiment discussed in this specification can be implemented with respect to any method or composition of the invention, and vice versa.

The use of the word “a” or “an” when used in conjunction with the terms “comprising,” “including,” “having,” or “containing,” or any variations of these terms, in the claims and/or the specification may mean “one,” but it is also consistent with the meaning of “one or more,” “at least one,” and “one or more than one.”

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### Example

To illustrate the advantages of the methods and compositions of the present invention, serial cell counts observed after refrigerated storage of microalgae cultures propagated using the system with oxygen nanobubbles (“Treated”) and without oxygen nanobubbles (“Untreated”) were compared. Three representative experiments are described in the following Table 1, two with paired controls (A and B), and the third without an untreated control (C). For the experiments, algae from a monoculture of Chlorella vulgaris were used. The monoculture of Chlorella vulgaris was purchased from the University of Texas phycology laboratory and has been propagated since under standard conditions for growing algae. As culture medium, commercially available Guillard's/F/₂ medium was used.

TABLE 1
Algae cell counts at the time of harvest and later, after a period
of dark, refrigerated storage. This compares algae grown using
standard commercial methods to EnSoil Algae, grown with proprietary
technology that allows continued algae growth while in storage.

| | | |
|---|---|---|
| Cell count | Cell count | |
| at harvest | at follow-up | Cell count: |
| (millions/mL) | (millions/mL) | % change |

A. Paired samples comparing cell count at harvest and 5 days later.
Untreated = normal growing method. Treated = EnSoil Algae using
proprietary growing technology

| | | | |
|---|---|---|---|
| Untreated | 12.5 million/mL | 5.2 | −56% |
| Treated | 12.8 | 12.4 | −3% |

B. Another experiment comparing cell count at harvest and 19 days later.

| | | | |
|---|---|---|---|
| Untreated | 15 | 8.4 | −44% |
| Treated | 16 | 16.8 | +5% |

C. A third experiment. Cell count of treated algae at harvest and
119 days later. (No untreated control for this experiment.)

| | | | |
|---|---|---|---|
| Treated | 16 | 23.3 | +46% |

As can be derived from table 1, the methods and compositions according to the invention allow for comparable or increased cell counts at the time of harvest, less decay in cell count after 5 days of harvest, and even an increase in cell count after 19 and, respectively, 119 days of harvest. This is attributed to mixotrophic algae that are able to switch from autotrophism to heterotrophism. Mixotrophy is not induced in untreated samples. The mixotrophic algae are able to switch from autotrophism to heterotrophism and are thus able to survive in the dark, e.g. when being stored. The controls in the experiments according to A. and B. of Table 1 show an almost 50% decline in cell count. The algae compositions of the present invention, however, advantageously, decline to a much lesser degree, and even show growth, when untreated controls continue to decline in cell count. The Experiments according to B. and C. of Table 1 show, that the algae even continue to propagate indicating that they are healthy and live.

### BRIEF DESCRIPTION OF THE DRAWINGS AND TABLE

FIG. 1 is a schematic representation of the growing system illustrating the improvement of the system used to grow microalgae using a PBR growing system comprising addition of the NBG and attached oxygen concentrator.

FIG. 2 illustrates a preferred arrangement for eight PBRs (10) plumbed in parallel with valves (25) separating each PBR from others to prevent contamination for a growing system. The valves (25) are also operated to allow filling and harvest of microalgae from individual PBRs from piping at the base of the PBR (45).

FIG. 3 is a detailed drawing of a preferred embodiment of a growing system (90) improved by addition of a nanobubble generator (NBG) (50) with an attached oxygen concentrator (55), such that an improved method can be used for growth of microalgae. As shown, the growing system (90) uses two holding tanks (30), each having a capacity of 500 gallons (approximately 2000 liters). The holding tanks (30) are positioned higher than the PBRs (10) to allow gravity-feeding of the sterile water to the PBRs (10), after passing it through the nanobubble generator (NBG) (50) with an attached oxygen concentrator (55), to saturate the water with oxygen nanobubbles. Each PBR (10) has a close-fitting, removable lid (15) that can be opened to access the inside of the PBR (40), which allows for the addition of inorganic nutrient solution to the water as well as the algae inoculant once each PBR used in the system is filled. The PBRs (10) in FIG. 3 are shaded to show growth medium (40) containing microalgae, some of which are at a lower level reflecting recent harvest before replenishment of sterilized, hyperoxygenated water and inorganic nutrient solution and reinoculation.

### DETAILED DESCRIPTION OF THE FIGURES AND ILLUSTRATIVE EMBODIMENTS

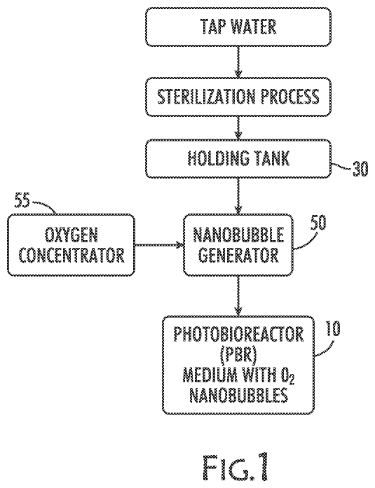

Referring to the drawings, FIG. 1 is a schematic representation of a preferred embodiment of a growing system improved by the addition of an NBG with an attached oxygen concentrator. Preparation of growth medium used to fill each PBR for growing microalgae using the improved method begins with sterilization of water drawn from a municipal source or a well and is not distilled water. The water is sterilized for use in the growing system, which is done by the introduction of ozone to the holding tank, as sometimes used in the commercial production of microalgae.

Sterilized water from the holding tank is gravity fed through a nanobubble generator. An oxygen concentrator is attached to the nanobubble generator to supply oxygen for the creation of oxygen nanobubbles to saturate the sterilized water. At sea level and room temperature, the oxygen content of water in the holding tank is 7 ppm. After addition of oxygen nanobubbles, the oxygen content of water reaching the PBR is at least 50 ppm, with an oxygen saturation of approximately 500%. Following this hyperoxygenation process, the water is then pumped to at least one PBR, a cylindrical container constructed of translucent fiberglass.

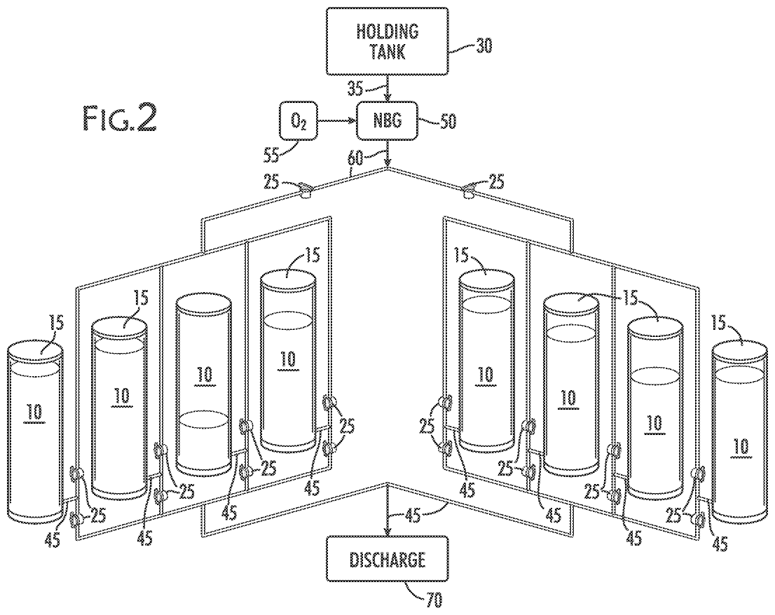

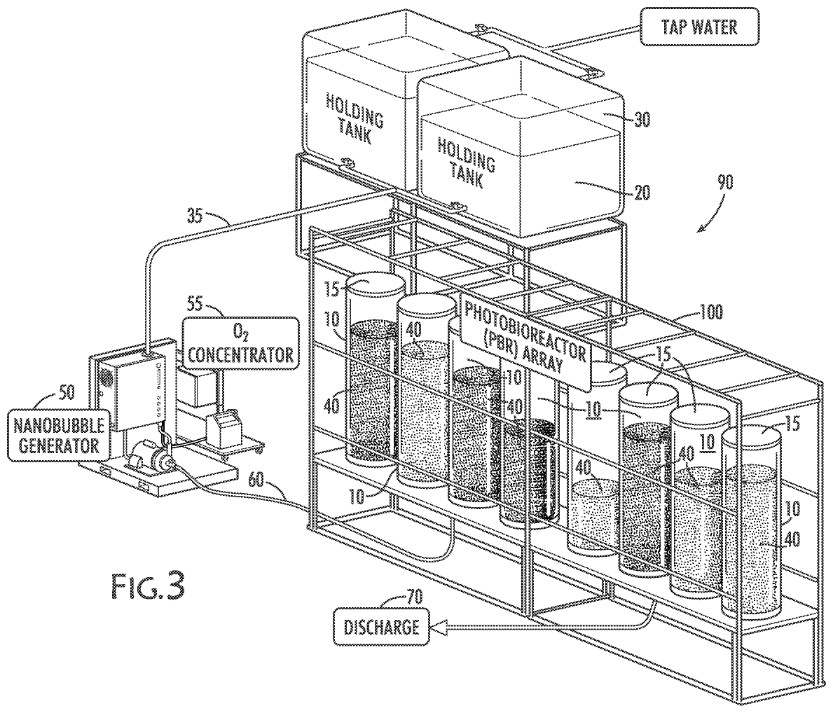

FIG. 2 is detailed depiction of eight PBRs (10) plumbed in parallel for use in a growing system. The growing system in depicted in FIG. 3. The PBRs (10) as illustrated each have a capacity of 80 gallons (300 liters). Each PBR (10) carries a fitted, removable lid (15) constructed of the same translucent material, preferably fiberglass, which prevents contamination of the contents by airborne particles or dust, but which can also be removed for access into the PBR.

FIG. 3 is a detailed drawing of a preferred embodiment of a growing system (90) improved by the addition of a NBG (50) with an attached oxygen concentrator (55). FIG. 3 does not depict the plumbing system described in FIG. 2. In this embodiment tap water fills at least one holding tank (30) placed at a higher elevation than the top of the PBRs (10) used in the growing system (90). As described in FIG. 1, sterilized water is gravity-fed from the holding tank (30) through a valve (25) carried on the tank and connected to pipe (35) that is attached to a nanobubble generator or NBG (50). An oxygen concentrator (55) is attached (60) to the NBG (50) to add oxygen to generate oxygen-filled nanobubbles that are injected into the water as it passes through the NBG (50). After receiving the oxygen nanobubbles, the hyperoxygenated water is pumped through tubing (60) connected to the plumbing system illustrated in FIG. 2. By opening and closing the valves the plumbing can be used to fill or drain individual PBRs (10) comprising the production system (90). The plumbing in the growing system (90) consists of polyethylene and stainless steel tubing. As shown in FIG. 2, the plumbing can be arranged to transport the hyperoxygenated water from the nanobubble generator to multiple PBRs plumbed in parallel and separable by valves (25) installed onto the piping that can be opened and closed to fill or drain any individual PBR (10). Both FIGS. 2 and 3 depict PBRs (10) filled with growth medium at different levels of harvest.

As illustrated in FIG. 2, the hyperoxygenated water is introduced through piping connected near the base of each PBR (45). After filling the PBR, inorganic nutrient solution, known in the industry as f/2 or F/2, is added to the sterilized, hyperoxygenated water from the top of the PBR tank by removing the lid (15). At this point the hyperoxygenated growth medium is complete and ready for introduction of algae inoculant.

Immediately after creating the growth medium (40) an algae inoculant, 5 gallons (20 liters) of Chlorella vulgaris grown to a cell count of 6-8 million cells/mL is poured into the PBR by removing the lid (15). The Chlorella vulgaris strain currently used by the authors was originally purchased from the phycology laboratory at the University of Texas and has been propagated using standard methods.

While not shown in the figures, tubular LED grow lights having a wave length of 440 nM are positioned vertically around each PBR a regular intervals to provide light for photosynthesis. This lighting is set to cycle on for 16 hours and off for 8 hours to simulate a 24-hour day. The inoculated growth medium (40) in the PBR (10) is continuously mixed by introduction of ambient air through an aquarium stone bubbler positioned inside the PBR at the base (not shown in the figures). The stone bubbler is attached by tubing to an external pump mounted outside of the PBR that contains a filter and an air dryer (not shown in the figures). The delivery of ambient air also provides carbon dioxide needed for photosynthesis.

In reference to FIG. 3, growth of the microalgae is monitored over time by drawing samples from the selected PBR (10) from the discharge line (70) attached to the piping (35) connected to each PBR (10) comprising the system (90). Specifically, the valve (25) carried on piping (45) connected to the base of the PBR (10) is opened so that the growth medium and microalgae (40) can be drained from the PBR through the piping (35) for sampling and for harvest. Cells are counted with a hemocytometer or automatic cell counter. Once the cell count in the growth medium (40) exceeds 12 million cells/mL, the culture is a finished “algae concentrate” ready for harvesting, again by opening the valve (25) to drain the desired volume of the through the piping at the base (45) of the PBR (10). A discharge line (70) is connected to the piping to be used for filling polyethylene containers that are placed into refrigerated storage at 6° C. The containers can be any size.

All of the algae concentrate can be harvested from a single PBR tank, or the harvest may be partial, typically drawing 10%-20% of the volume from the PBR. After partial harvest, the volume is replaced with new growth medium (i.e., hyperoxygenated, sterile water to which additional inorganic nutrients may be added). Partial harvest does not require re-inoculation with additional algae culture since the algae remaining in the PBR continue to grow. Typically, the cell count in the PBR recovers to the pre-harvest level in 4-5 days. Thus, with partial harvest, as much as 20% of the PBR's volume can be taken at 5-day intervals. In this case, a PBR can remain in active service for as long as 5 months.

The size of PBRs (10) can vary. The preferred embodiment of the system (90) includes PBRs constructed to 6 feet tall with a capacity of 360 gallons (1350 liters). FIGS. 2 and 3 show the preferred assembly of multiple PBR tanks (10), with FIG. 3 illustrating a photobioreactor array (55) involving eight PBRs (10) held on a rack (100). The rack (100) is constructed of metal, preferably extruded aluminum. The number of individual PBRs that may be used in the PBR array is limited only by available space of the building housing the production system (90). The number of holding tanks can be expanded as well, again depending upon available space of the building.

It is intended that the scope of the present invention include all modifications that incorporate its principal design features, and that the scope and limitation of the present invention are to be determined by the scope of the appended claims and their equivalents. It also should be understood, therefore, that the inventive concepts herein described are interchangeable and/or they can be used together in still other permutations of the present invention, and that other modifications and substitutions will be apparent to those skilled in the art of propagation of microalgae using photobioreactors from the foregoing description of the preferred embodiments without departing from the spirit or scope of the present invention.

1. A method of producing an algae composition that comprises viable algae and a liquid, comprising the step(s) of
adding oxygen to the liquid to provide for a hyperoxygenated liquid;
culturing the algae n the hyperoxygenated liquid at least temporarily under light exposure to allow propagation of the algae.

2. The method of claim 1 comprising the steps of providing a liquid;
adding oxygen to the liquid to provide for a hyperoxygenated liquid;
inocculating the hyperoxygenated liquid with algae;
culturing the algae in the hyperoxygenated liquid at least temporarily under light exposure to allow propagation of the algae.

3. The method of claim 1 comprising the steps of
providing a liquid comprising algae;
adding oxygen to the liquid to provide for a hyperoxygenated liquid and/or adding hyperoxygenated liquid;
culturing the algae in the hyperoxygenated liquid at least temporarily under light exposure to allow propagation of the algae.

4. The method of any one of claims 1 to 3, wherein the viable cell count in the liquid is between 1 and 20 million cells/mL preferably between 2 and 18 million cells/mL more preferably between 5 and 15 million cells/mL, even more preferably between 7 and 14 million cells/mL, still more preferably between 10 and 13 million cells/mL, most preferably between 11 and 13 million cells/mL.

5. The method of any one of claims 1 to 4, wherein a decay in viable cell count is at most 90%, preferably at most 80%, more preferably at most 70%, even more preferably at most 60%, still more preferably at most 50%, still more preferably at most 40%, still more preferably at most 30%, still more preferably at most 20%, still more preferably at most 10%, still more preferably at most 5% of the decay in viable cell count of a control culture of the same algae which is not hyperoxygenated within the same period of time.

6. The method of anyone of claims 1 to 5, wherein there is at most 10%, preferably at most 8%, more preferably at most 6%, even more preferably at most 4%, still more preferably at most 2%, still more preferably at most 1%, most preferably no decay in viable cell count within a time period of
(i) between 1 and 24 months, preferably between 1 and 20 months, more between 1 and 18 months, even more preferably between 2 and 15 months, still more preferably between 2 and 12 months, even more preferably between 2 and 10 months, even more preferably between 3 and 9 months, most preferably between 4 and 8 months; or
(ii) between 1 and 3D days, preferably 1 and 25 days, more preferably 1 and 15 days, even more preferably 1 and 10 days, most preferably 4 and 6 days; or
(iii) at least 1 month, preferably at least 2 months, more preferably at least 3 months, even more preferably at least 4 months, still more preferably at least 5 months, even more preferably at least 6 months, still more preferably at least 7 months, most preferably at least 8 months;
when compared to a control culture of the same algae which is not hyperoxygenated.

7. The method of any one of claims 1 to 6, wherein the viably cell count increases by at least 1%, preferably at least 5%, more preferably at least 10%, even more preferably at least 20%, still more preferably at least 30%, most preferably at least 40% within a time period of
(i) between 1 and 30 days, preferably 5 and 25 days, more preferably 10 and 25 days, even more preferably 15 and 25 days, most preferably 18 and 22 days, or
(ii) of between 1 and 24 months, preferably between 1 and 20 months, more between 1 and 18 months, even more preferably between 2 and 15 months, still more preferably between 3 and 12 months, even more preferably between 3 and 10 months, even more preferably between 3 and 8 months, still more preferably between 3 and 6 months, most preferably between 3 and 4 months,
when compared to a control culture of the same algae which is not hyperoxygenated.

8. The method of any one of claims 1 to 7, wherein the viable algae can be stored at a temperature of between 0° C. and 10° C., preferably between 2° C. and 8° C., more preferably between 5° C. and 7° C. for at least 1 month, preferably at least 2 months, more preferably at least 4 months, even more preferably at least 5 months, still more preferably at least 6 months, even more preferably at least 7 months, even more preferably at least 8 months, most preferably as long as 18 months and/or wherein the viable algae can be stored at room temperature, preferably at a temperature of between 10° C. and 30° C., preferably between 15° C. and 25° C., more preferably between 18° C. and 22° C. for at least 1 week, preferably at least 2 weeks, more preferably at least 3 weeks, even more preferably at least 4 weeks, still more preferably at least 5 weeks, even more preferably at least 8 weeks, still more preferably at least 7 weeks, most preferably at least 8 weeks, in particular at least 2 months without significant decay in viable cell count compared to the viable cell count at the time of harvesting the algae.

9. The method of any one of claims 1 to 8, wherein oxygen is added only once.

10. The method of any one of claims 1 to 9, comprising more than one step of adding oxygen to the liquid to provide for a hyperoxygenated liquid and/or adding hyperoxygenated liquid.

11. The method of any one of claims 1 to 8, wherein oxygen is added continuously to the liquid.

12. The method of anyone of claims 1 to 11, wherein the method is carried out in a photobioreactor.

13. The method of anyone of claims 1 to 12, wherein exposing the algae in the liquid to light is carried out for at least 1 to 24 hours at 1 to 12 hour intervals, preferably at least 16 hours at 8 hour intervals.

14. The method of any one of claims 1 to 13, wherein exposing the algae in the liquid to light is carried out at a wavelength of 200 nm to 800 nm, preferably 250 nm to 650 nm, more preferably 300 to 550 nm, even more preferably 400 to 500 nm, most preferably 440 nm.

15. The method of any one of claims 1 to 14, wherein exposing the algae in the liquid to light is carried out at a power of 1 to 20 W, preferably 6 to 20 W, more preferably 10 to 20 W, even more preferably 10 to 15 W, most preferably 13 W.

16. The method of any one of claims 1 to 15, wherein exposing the algae in the liquid to light is carried out at a light intensity of between 1,000 and 20,000 lux, preferably 5,000 and 15,000 lux, more preferably of between 8,000 and 12,000 lux.

17. The method of any one of claims 1 to 16, wherein the method further comprises
(i) monitoring the viable cell count; and/or
(ii) harvesting the algae; and/or
(iii) concentrating the harvested algae; and/or
(iv) storing the algae.

18. The method of any one of claims 1 to 17, wherein the viable algae are stored (i) at a temperature of between 0° C. and 10° C., preferably between 2° C. and 8° C., more preferably between 5° C. and 7° C.; and/or
(ii) at a temperature of between 10° C. and 30° C., preferably between 15° C. and 25° C., more preferably between 18° C. and 22° C.; and/or
(iii) in the dark.

19. The method of any one of claims 1 to 18, wherein oxygen is added to the liquid by supplying oxygen nanobubbles to the liquid.

20. The method of anyone of claims 1 to 19, wherein the liquid has an oxygen level of at least 20 ppm, preferably at least 25 ppm, more preferably at least 30 ppm, even more preferably at least 35 ppm, still more preferably at least 40, even more preferably at least 45 ppm, most preferably at least 50 ppm.

21. The method of anyone of claims 1 to 20, wherein the liquid has an oxygen saturation of at least 100%, preferably of at least 200%, more preferably at least 300%, even more preferably at least 400%, most preferably at least 500%.

22. The method of any one of claims 2 to 21, wherein the hyperoxygenated liquid which is inoculated with the algae has an oxygen level of at least 50 ppm and/or an oxygen saturation of at least 500%.

23. The method of anyone of claims 1 to 22, wherein the liquid is an aqueous solution, preferably selected from water, fresh water, sea water, sterilized water, culture medium and/or buffer.

24. The method of any one of claims 1 to 17, wherein the liquid is a culture medium, preferably the culture medium comprising
(a) water, preferably sterilized water;
(b) nitrate, preferably an alkalimetal salt thereof, more preferably sodium nitrate;
(c) dihydrogenphosphate preferably an alkalimetal salt thereof, more preferably sodium dihydrogenphosphate;
(d) silicate, preferably an alkalimetal salt thereof, more preferably sodium silicate;
(e) one or more trace metals, preferably inorganic salts thereof, more preferably the trace metals are selected from cobalt, copper, iron, manganese, molybdenmum and/or zink; and/or
(f) one or more vitamins, preferably selected from vitamin B₁₂, biotin, and/or thiamine.

25. The method of any one of claims 1 to 24, wherein there is no externally added carbohydrate present in the liquid.

26. The method of anyone of claims 1 to 25, wherein no additional carbohydrate is added to the liquid to induce heterotrophic metabolism in the algae.

27. The method according to any one of claims 1 to 26, operated in batch mode, fed-batch mode, semi-continuous mode or continuous mode.

28. The method of anyone of claims 1 to 27, wherein the algae are capable of mixotrophic metabolism, preferably wherein the algae are capable of both autotrophic and heterotrophic metabolism, even more preferably the algae have autotrophic metabolism when grown under light exposure and are capable of heterotrophic metabolism in the absence of light.

29. The method of any one of claims 1 to 28, wherein the algae are obligate mixotroph, obligate autotroph and facultative heterotroph, facultative autotroph and obligate heterotroph, and/or facultative mixotroph.

30. The method of anyone of claims 1 to 29, wherein the algae are selected from the group of unicellular algae, preferably green algae, more preferably the algae are Chlorella, even more preferably the algae are Chlorella vulgaris.

31. A composition comprising viable algae and a liquid, wherein the composition is obtainable by a method according to any one of claims 1 to 30.

32. A composition comprising viable algae and a liquid, wherein the liquid is hyperoxygenated.

33. The composition of claim 31 or 32, wherein the liquid comprises oxygen nanobubbles, preferably wherein the liquid is saturated with oxygen nanobubbles.

34. The composition of anyone of claims 31 to 33, wherein the liquid has an oxygen level of at least 20 ppm, preferably at least 25 ppm, more preferably at least 30 ppm, even more preferably at least 35 ppm, still more preferably at least 40, even more preferably at least 45 ppm, most preferably at least 50 ppm.

35. The composition of any one of claims 31 to 34, wherein the liquid has an oxygen saturation of at least 200%, preferably at least 300%, more preferably at least 400%, most preferably at least 500%.

36. The composition of any one of claims 31 to 35, wherein the liquid is selected from an aqueous solution, preferably selected from water, fresh water, sea water, sterilized water, culture medium and/or buffer.

37. The composition of any one of claims 31 to 36, wherein the liquid is a culture medium comprising
(a) water, preferably sterilized water;
(b) nitrate, preferably an alkalimetal salt thereof, more preferably sodium nitrate;
(c) dihydrogenphosphate preferably an alkalimetal salt thereof, more preferably sodium dihydrogenphosphate;
(d) silicate, preferably an alkalimetal salt thereof, more preferably sodium silicate;
(e) one or more trace metals, preferably inorganic salts thereof, more preferably the trace metals are selected from cobalt, copper, iron, manganese, molybdenmum and/or zink; and/or
(f) one or more vitamins, preferably selected from vitamin B₁₂, biotin, and/or thiamine.

38. The composition of any one of claims 31 to 37, wherein there is no externally added carbohydrate present in the liquid.

39. The composition of any one of claims 31 to 38, wherein no additional carbohydrate is added to the liquid.

40. The composition of any one of claims 31 to 39, wherein the algae are capable of mixotrophic metabolism, preferably wherein the algae are capable of both autotrophic and heterotrophic metabolism, even more preferably the algae have autotrophic metabolism when grown under light exposure and are capable of heterotrophic metabolism in the absence of light.

41. The composition of any one of claims 31 to 40, wherein the algae are obligate mixotroph, obligate autotroph and facultative heterotroph, facultative autotroph and obligate heterotroph, and/or facultative mixotroph.

42. The composition of any one of claims 31 to 41, wherein the algae are selected from the group of unicellular algae, preferably green algae, more preferably the algae are Chlorella, even more preferably the algae are Chlorella vulgaris.

43. The composition of any one of claims 31 to 42, wherein the viable cell count in the liquid is between 1 and 20 million cells/mL, preferably between 2 and 18 million cells/mL, more preferably between 5 and 15 million cells/mL, even more preferably between 7 and 14 million cells/mL, still more preferably between 10 and 13 million cells/mL, most preferably between 11 and 13 million cells/mL.

44. The composition of any one of claims 31 to 43, wherein a decay in viable cell count is at most 90%, preferably at most 80, more preferably at most 70%, even more preferably at most 60%, still more preferably at most 50%, still more preferably at most 40%, still more preferably at most 30%, still more preferably at most 20%, still more preferably at most 10%, still more preferably at most 5%, when compared to the decay in viable cell count of a control culture of the same algae which is not hyperoxygenated in the same period of time.

45. The composition of any one of claims 31 to 44, wherein there is at most 10%, preferably at most 8%, more preferably at most 6%, even more preferably at most 4%, still more preferably at most 2%, still more preferably at most 1%, most preferably no decay in viable cell count within a time period of
(i) between 1 and 24 months, preferably between 1 and 20 months, more between 1 and 18 months, even more preferably between 2 and 15 months, still more preferably between 2 and 12 months, even more preferably between 2 and 10 months, even more preferably between 3 and 6 months, most preferably between 3 and 5 months; or
(ii) between 1 and 30 days, preferably 1 and 25 days, more preferably 1 and 15 days, even more preferably 1 and 10 days, most preferably 4 and 6 days; or
(iii) at least 1 month, preferably at least 2 months, more preferably at least 3 months, even more preferably at least 4 months, still more preferably at least 5 months, even more preferably at least 6 months, still more preferably at least 7 months, most preferably at least 8 months;
when compared to a control culture of the same algae which is not hyperoxygenated.

46. The composition of any one of claims 31 to 44, wherein the viably cell count increases by at least 1%, preferably at least 5%, more preferably at least 10%, even more preferably at least 20%, still more preferably at least 30%, most preferably at least 40% within a time period of
(i) between 1 and 30 days, preferably 5 and 25 days, more preferably 10 and 25 days, even more preferably 15 and 25 days, most preferably 18 and 22 days, or
(ii) between 1 and 24 months, preferably between 1 and 20 months, more between 1 and 18 months, even more preferably between 2 and 15 months, still more preferably between 3 and 12 months, even more preferably between 3 and 10 months, even more preferably between 3 and $ months, still more preferably between 3 and 8 months, most preferably between 3 and 4 months, when compared to a control culture of the same algae which is not hyperoxygenated.

47. Use of the composition of any one of claims 31 to 46 for improving plant growth.

48. The use of claim 47, wherein the plants are selected from fruits, vegetables and/or crops, preferably the crops are agricultural plants grown for food or fiber.

49. The use of claim 48, wherein the fruits are selected from fruit-bearing trees, berry bushes, and/or pineapples.

50. The use of claim 48, wherein the vegetables are selected from garden vegetables, preferably tomatoes, potatoes, cucumbers, pepper, carrots, squash, and/or pumpkin.

51. The use of claim 48, wherein the crops are selected from vegetable crops, sugar beets, corn, beans, hay, peanuts, cotton, hemp and/or tobacco.

52. A method of maintaining or improving soil fertility and/or for improving plant growth comprising the step(s) of applying the composition of any one of claims 31 to 46 to soil and/or plants.

53. The method of claim 52, wherein the soil is agricultural land, pastureland, a playing field, a golf course, and/or a civic green space.

54. The method of claim 52 or 53, comprising the step of diluting the composition of any one of claims 25 to 37 before applying the composition to soil and/or plants.

55. The method of any one of claims 52 to 54, wherein the composition is applied in an amount of between 10 and 100,000 cells per sq ft, preferably between 100 and 90,000 cells per sq ft, preferably between 1,000 and 80,000 cells per sq ft, more preferably between 10,000 and 70,000 cells per sq ft, still more preferably between 20,000 and 60,000 cells per sq ft, even more preferably between 30,000 and 55,000 cells per sq ft, still more preferably between 35,000 and 55,000 cells per sq ft, still more preferably between 40,000 and 55,000 cells per sq ft, even more preferably between 45,000 and 55,000 cells per sq ft, most preferably in an amount of 50,000 cells per sq ft.

56. A photobioreactor comprising means for supplying oxygen in the form of nanobubbles.

57. The photobioreactor of claim 56, wherein the means for supplying oxygen in the form of nanobubbles comprise means for providing oxygen, preferably an oxygen concentrator, and means for generating nanobubbles, preferably a nanobubble generator.

58. The photobioreactor of claim 57, wherein the means for providing oxygen are operably connected to the means for generating nanobubbles.

59. The photobioreactor of any one of claims 56 to 58, comprising one or more of
(a) one or more reaction vessels, preferably made from fiberglass;
(b) one or more reservoirs;
(c) one or more light sources, preferably LED light sources, more preferably a tubular LED grow light and/or LED bulbs;
(d) one or more means for supplying dissolved gas to the photobioreactor;
(e) an oxygen concentrator;
(f) piping; and/or
(g) one or more valves.

60. The photobioreactor of any one of claims 56 to 59, comprising a reaction vessel, preferably wherein the reaction vessel is a container characterized by one or more of the following:
(a) the container is liquid-impermeable;
(b) the container is cylindrically-shaped;
(c) the container has fixed side walls and bottom;
(d) the container has a removable lid; and/or
(e) the container is fabricated from a translucent material, preferably comprising fiberglass.

61. The photobioreactor of any one of claims 56 to 60, comprising a light source, preferably a tubular LED grow light and/or a LED bulb, more preferably wherein the light source is characterized by one or more of the following:
(a) a wavelength of 200 nm to 800 nm, preferably 250 nm to 650 nm, more preferably 300 to 550 nm, even more preferably 400 to 500 nm, most preferably 440 nm; and/or
(b) a light intensity of between 1,000 and 20,000 lux, preferably 5,000 and 15,000 lux, more preferably of between 8,000 and 12,000 lux; and/or
(c) a power of 1 to 20 W, preferably 5 to 20 W, more preferably 10 to 20 W, even more preferably 10 to 15 W, most preferably 13 W; and/or
(d) the light source being positioned vertically and equidistant around the one or more reaction vessels, preferably the light source being positioned at a distance of 0.5 to 50 cm, preferably 1 to 10 cm, more preferably 2 to 8 cm, even more preferably 5 to 6 cm, most preferably 5 cm from the one or more reaction vessels;
and/or
(e) the light source comprising a timer operable to switch on and off the light source, preferably the timer is set to cycle said light source on for at least 1 to 24 hours and off for at least 1 to 12 hours, preferably on for 16 hours and off for 8 hours.

62. The photobioreactor of any one of claims 56-61, comprising one or more means for supplying dissolved gas to the one or more reaction vessels which is not the means for supplying oxygen in the form of nanobubbles, preferably, wherein the one or more means for supplying dissolved gas to the one or more reaction vessels comprises a pump operable to push dissolved gas into the one or more reaction vessels, tubing for the dissolved gas to pass through, and a check valve, more preferably, wherein the one or more means for supplying dissolved gas to the one or more reaction vessels is an aquarium stone bubbler.

63. The photobioreactor of any one of claims 56 to 62, comprising piping, preferably wherein the piping is made from a polymer, preferably polyethylene, and/or stainless steel, preferably the piping is made from a combination of polyethylene and stainless steel.

64. The photobioreactor of any one of claims 56 to 63 comprising piping, preferably wherein the piping is adapted to provide for fluid connection of the of the photobioreactor's components.

65. A system comprising one or more photobioreactors according to any one of claims 56 to 64.

66. The system of claim 65 comprising at least two photobioreactors, preferably wherein the photobioreactors are in fluid connection.

67. The system of claim 66, wherein the photobioreactors are arranged in a photobioreactor array.

68. The system of claim 66 or 67, wherein the photobioreactors are connected in parallel.

69. The system of any one of claims 65 to 68, comprising valves, wherein the valves are operable to allow separation of said at least one photobioreactor from other photobioreactors by opening or closing said valves.

70. The system of any one of claims 65 to 69, wherein the one or more photobioreactors are in fluid connection with a nanobubble generator.

71. The system of any one of claims 65 to 70, comprising a reservoir in fluid connection with the one or more photobioreactors.

72. The system of any one of claims 65 to 71, comprising a discharge line operably connected to the one or more photobioreactors to remove liquid from the one or more photobioreactors, preferably the discharge line is made from polyethylene.
